SEQUENCE LISTING (21 sequences)

SEQ ID NO: 1 - protein, 331 aa, Mus musculus
MGESNGEAFLAFKTSASPTAPVTTNPMDETLPGSINIRILIPKLMIIIFGLVGLMGNAIVFWLLGFHLRRNAFSVYILNLALADFLFLLSSIIASTLFLLKVSYLSIIFHLCFNTIMMVVYITGISMLSAISTECCLSVLCPTWYRCHRPVHTSTVMCAVIWVLSLLICILNSYFCAVLHTRYDNDNECLATNIFTASYMIFLLVVLCLSSLALLARLFCGAGQMKLTRFHVTILLTLLVFLLCGLPFVIYCILLFKIKDDFHVLDVNLYLALEVLTAINSCANPIIYFFVGSFRHQLKHQTLKMVLQSALQDTPETAENMVEMSSNKAEP

SEQ ID NO: 2 - protein, 321 aa, Homo sapiens
MNQTLNSSGTVESALNYSRGSTVHTAYLVLSSLAMFTCLCGMAGNSMVIWLLGFRMHRNPFCIYILNLAAADLLFLFSMASTLSLETQPLVNTTDKVHELMKRLMYFAYTVGLSLLTAISTQRCLSVLFPIWFKCHRPRHLSAWVCGLLWTLCLLMNGLTSSFCSKFLKFNEDRCFRVDMVQAALIMGVLTPVMTLSSLTLFVWVRRSSQQWRRQPTRLFVVVLASVLVFLICSLPLSIYWFVLYWLSLPPEMQVLCFSLSRLSSSVSSSANPVIYFLVGSRRSHRLPTRSLGTVLQQALREEPELEGGETPTVGTNEMGA

SEQ ID NO: 3 - protein, 325 aa, Mus musculus
MDIDISSLGIYIIAPNGSSYTNSVDCFFKIQVMGFLSLIISPVGMVLNSTVLWFLGFQIRRNAFSVYILNLAGADFLFLHSQFLFYLLAIFPSIPIQIPLFFDMLTKFAYLSGLSILSTISIERCLCVMWPIWYRCQRPRHTSSVTCSLLWALSLLFALLDGMGCGLLFNSFDQSWCLKFDLIICAWSIVLFVVLCGSSLILLVRIFCGSQQIPVTRLYVTIALTVLFFLICCLPFGISWIIQWSETLIYVGFCDYFHEELFLSCINSCANPIIYFLVGFIRQRKFQQKSLKVLLQRAMEDTPEEENEDMGPSRNPEEFETVCSN

SEQ ID NO: 4 - protein, 330 aa, Homo sapiens
MDPTTPAWGTESTTVNGNDQALLLLCGKETLIPVFLILFIALVGLVGNGFVLWLLGFRMRRNAFSVYVLSLAGADFLFLCFQIINCLVYLSNFFCSISINFPSFFTTVMTCAYLAGLSMLSTVSTERCLSVLWPIWYRCRRPRHLSAVVCVLLWALSLLLSILEGKFCGFLFSDGDSGWCQTFDFITAAWLIFLFMVLCGSSLALLVRILCGSRGLPLTRLYLTILLTVLVFLLCGLPFGIQWFLILWIWKDSDVLFCHIHPVSVVLSSLNSSANPIIYFFVGSFRKQWRLQQPILKLALQRALQDIAEVDHSEGCFRQGTPEMSRSSLV

SEQ ID NO: 5 - DNA, 993 bp, Mus musculus
atgggggaaagcaatggtgaagcatttcttgcctttaagacctcagcctcaccaacagcaccagtgacaacaaatccaatggacgaaaccctccctggaagtatcaacattaggattctgatcccaaaattgatgatcatcatcttcggactggtcggactgatgggaaacgccattgtgttctggctcctgggcttccacttgcgcaggaatgccttctcagtctacatcctaaacttggccctggctgacttccttttcctcctcagtagtatcatagcttccaccctgtttcttctcaaagtttcctacctcagcatcatctttcacttgtgctttaacaccattatgatggttgtctacatcacagggataagcatgctcagtgccatcagcactgagtgctgcctgtctgtcctgtgccccacctggtatcgctgccaccgtccagtacatacatcaactgtcatgtgtgctgtgatctgggtcctatccctgttgatctgcattctgaatagctatttctgtgctgtcttacataccagatatgataatgacaatgagtgtctggcaactaacatctttaccgcctcgtacatgatatttttgcttgtggtcctctgtctgtccagcctggctctgctggccaggttgttctgtggcgctgggcagatgaagcttaccagatttcatgtgaccatcttgctgacccttttggtttttctcctctgcgggttgccctttgtcatctactgcatcctgttattcaagattaaggatgatttccatgtattagatgttaatctttatctagcattagaagtcctgactgctattaacagctgtgccaaccccatcatctacttcttcgtgggctctttcagacatcagttgaagcaccagaccctcaaaatggttctccagagtgcactgcaggacactcctgagacagctgaaaacatggtagagatgtcaagtaacaaagcagagcct

SEQ ID NO: 6 - DNA, 966 bp, Homo sapiens
atgaaccagactttgaatagcagtgggaccgtggagtcagccctaaactattccagagggagcacagtgcacacggcctacctggtgctgagctccctggccatgttcacctgcctgtgcgggatggcaggcaacagcatggtgatctggctgctgggctttcgaatgcacaggaaccccttctgcatctatatcctcaacctggcggcagccgacctcctcttcctcttcagcatggcttccacgctcagcctggaaacccagcccctggtcaataccactgacaaggtccacgagctgatgaagagactgatgtactttgcctacacagtgggcctgagcctgctgacggccatcagcacccagcgctgtctctctgtcctcttccctatctggttcaagtgtcaccggcccaggcacctgtcagcctgggtgtgtggcctgctgtggacactctgtctcctgatgaacgggttgacctcttccttctgcagcaagttcttgaaattcaatgaagatcggtgcttcagggtggacatggtccaggccgccctcatcatgggggtcttaaccccagtgatgactctgtccagcctgaccctctttgtctgggtgcggaggagctcccagcagtggcggcggcagcccacacggctgttcgtggtggtcctggcctctgtcctggtgttcctcatctgttccctgcctctgagcatctactggtttgtgctctactggttgagcctgccgcccgagatgcaggtcctgtgcttcagcttgtcacgcctctcctcgtccgtaagcagcagcgccaaccccgtcatctacttcctggtgggcagccggaggagccacaggctgcccaccaggtccctggggactgtgctccaacaggcgcttcgcgaggagcccgagctggaaggtggggagacgcccaccgtgggcaccaatgagatgggggcttga

SEQ ID NO: 7 - DNA, 978 bp, Mus musculus
atggatatagacatttcgagcctgggcatttacatcatagcaccgaatggaagcagctacactaatagtgttgattgtttcttcaaaatccaagtcatgggttttctttccctcatcatttcccctgttgggatggtattaaattccacagtgctgtggtttctgggcttccagatacgtaggaatgccttctctgtctacatcctcaacctggccggggctgactttctcttcctgcactctcagtttttattttaccttcttgctatttttccctccattcctatccagatccctctcttttttgatatgttgacaaaatttgcatatctttctgggctgagcattctcagcaccattagcattgagcgctgcctgtgtgtcatgtggcccatctggtaccgctgtcaaagaccaagacacacatcatctgtaacctgttccttgctttgggctttgtccctgttgtttgctcttctggatgggatgggatgtggcttactgtttaatagttttgaccagtcttggtgtttgaaatttgatttaatcatttgtgcgtggtcaattgttttatttgtggttctctgtgggtccagtctcatcctacttgttaggatcttctgtggctcccagcagatccctgtgaccaggctgtatgtgaccattgcactcacagtgttattcttcctaatctgctgtcttccctttggaatctcctggatcatccaatggagtgaaactttgatatatgttggattttgtgattattttcacgaggaactattcctatcctgtattaacagctgtgccaaccctatcatttacttccttgttggttttattcgtcagcgaaagttccaacagaagtctctgaaggtgcttcttcaaagagcgatggaggacactcctgaagaagaaaatgaagacatgggtccttcaagaaatccagaagaatttgaaacagtctgtagcaactga

SEQ ID NO: 8 - DNA, 1770 bp, Homo sapiens
aatcagtgaacatgcagcaggtgctcaagtcttgtttttgtttccaggggcaccagtggaggttttctgagcatggatccaaccaccccggcctggggaacagaaagtacaacagtgaatggaaatgaccaagcccttcttctgctttgtggcaaggagaccctgatcccggtcttcctgatccttttcattgccctggtcgggctggtaggaaacgggtttgtgctctggctcctgggcttccgcatgcgcaggaacgccttctctgtctacgtcctcagcctggccggggccgacttcctcttcctctgcttccagattataaattgcctggtgtacctcagtaacttcttctgttccatctccatcaatttccctagcttcttcaccactgtgatgacctgtgcctaccttgcaggcctgagcatgctgagcaccgtcagcaccgagcgctgcctgtccgtcctgtggcccatctggtatcgctgccgccgccccagacacctgtcagcggtcgtgtgtgtcctgctctgggccctgtccctactgctgagcatcttggaagggaagttctgtggcttcttatttagtgatggtgactctggttggtgtcagacatttgatttcatcactgcagcgtggctgatttttttattcatggttctctgtgggtccagtctggccctgctggtcaggatcctctgtggctccaggggtctgccactgaccaggctgtacctgaccatcctgctcacagtgctggtgttcctcctctgcggcctgccctttggcattcagtggttcctaatattatggatctggaaggattctgatgtcttattttgtcatattcatccagtttcagttgtcctgtcatctcttaacagcagtgccaaccccatcatttacttcttcgtgggctcttttaggaagcagtggcggctgcagcagccgatcctcaagctggctctccagagggctctgcaggacattgctgaggtggatcacagtgaaggatgcttccgtcagggcaccccggagatgtcgagaagcagtctggtgtagagatggacagcctctacttccatcagatatatgtggctttgagaggcaactttgcccctgtctgtctgatttgctgaactttctcagtcctgattttaaaacagttaagagagtccttgtgaggattaagtgagacagtgcctatgaaacaaacactaagtgcagtgtctctggaactgccttactcacaggcttccaccacagccctatgagagctttgccaactctgcggtccatgactgttcccacttttaatgaatcctacctttcgcagaaggctgaaagcagggcagaaaagatctacatttctttggacactgcacttgatagggactcaaagaatgttatatttttaattaatttctttttctcttccgtacaatttctgtctcaacaaaattagaagaattaaatttaaaactagctccaaaagagcagccgtctttcattttggcagaccttagaatatccccctagcttaataaatctttgttgaatggcttaatgaatgaataaactggttaatgtttaagttaaacctctgaaaagtctccatttaccagatttgagtcactaaatttattgctttcactacttttgaattcacctctccgttatgaccctaaatcacaatctccattctgactgttgatggactcctctcagtcattaagggtggccatgggtcatggcctgagtcatttc

SEQ ID NO: 9 - DNA, 24 bp, Mus musculus
gaggcatgtcaaatcagtaagctg

SEQ ID NO: 10 - DNA, 20 bp, Mus musculus
ggtggctttggagtgagcac

SEQ ID NO: 11 - DNA, 19 bp, Mus musculus
atcaacgggaagcccatca

SEQ ID NO: 12 - DNA, 20 bp, Mus musculus
gacatactcagcaccggcct

SEQ ID NO: 13 - DNA, 21 bp, Homo sapiens
ttcttctgctttgtggcaagg

SEQ ID NO: 14 - DNA, 21 bp, Homo sapiens
gaaaaggatcaggaagaccgg

SEQ ID NO: 15 - DNA, 653 bp, Mus musculus
  misc_feature 458, 482, 492, 523, 532, 534, 536, 540, 543, 547, 551,552, 556, 560, 567, 576, 580, 584, 587, 592, 595, 602, 603, 613,619, 629, 633, 641: n = A,T,C or G
tattcaggaggggtagaaaaactaagttagtggagagatgttgaagttgttggtggaccactgaggcatgtcaaatcagtaagctgagagaagagtggacagtggtcaagtgcagcagggcagtgctcactccaaagccacctctgaggtccaggtagaggctcttcatcaaggctctgctttgttacttgacatctctaccatgttttcagctgtctcaggagtgtcctgcagtgcactctggagaaccattttgagggtctggtgcttcaactgatgtctgaaagagcccacgaagaagtagatgatggggttggcacagctgttaatagcagtcaggacttctaatgctagataaagattaacatctaatacatggaaatcatccttaatcttgaataacaggatgcagtagatgacaaagggcacccgcagaggagaaaaaccaaaagggtcancaagatgggcacatgaaatctggnaagctttatntgccccagcgccacaaaaacaacctggccanaaaaaaccngngntggncangacngggnncccncccnccaaaanttttttttntttnctgnccnggggnggnccttttnnaaagcccatntttccnaccacccctngggngggggccntttttttttggg

SEQ ID NO: 16 - DNA, 22 bp, Homo sapiens
catgggtgtgaaccatgagaag

SEQ ID NO: 17 - DNA, 21 bp, Homo sapiens
ctaagcagttggtggtgcagg

SEQ ID NO: 18 - DNA, 25 bp, Homo sapiens
tcttaaccaccagatcattccttct

SEQ ID NO: 19 - DNA, 20 bp, Homo sapiens
ggatactgcgagcaaatggg

SEQ ID NO: 20 - DNA, 24 bp, Homo sapiens
ggagtcagccctaaactattccag

SEQ ID NO: 21 - DNA, 20 bp, Homo sapiens
aggtaggccgtgtgcactgt

(19) United States
(12) Patent Application Publication
(10) Pub. No.: US 2002/0116724 A1
(43) Pub. Date: Aug. 22, 2002

(54) Polypeptides or nucleic acids encoding these of a family of G-protein coupled receptors and their use for the diagnosis or treatment of disorders, for example skin disorders and their use for the identification of pharmacologically active substances

(76) Inventors: Eckhard Wolf, Oberschleissheim (DE); Sabine Werner, Zurich (CH); Jorn-Peter Halle, Penzberg (DE); Johannes Regenbogen, Martinsried (DE); Andreas Goppelt, Munchen (DE)

Correspondence Address: Karen L. Elbing, Ph.D., Clark & Elbing LLP, 101 Federal Street, Boston, MA, 02110-2214, US

(21) Appl. No.: 09/920,068

(22) Filed: Aug. 1, 2001

Related U.S. Application Data
(60) Provisional application No. 60/229,501, filed on Aug. 31, 2000.

(30) Foreign Application Priority Data
Aug. 4, 2000 (DE) 10038111.1

(51) Int. Cl. A01K 67/00; C07H 21/04; C12P 21/02; C12N 5/06; C07K 14/705

(52) U.S. Cl. 800/8; 435/325; 435/320.1; 530/350; 536/23.5

(57) ABSTRACT

Polypeptides or nucleic acids encoding the polypeptides of a family of G-protein coupled receptors and their for use for prevention, diagnosis and/or treatment of disorders, for example skin disorders, and/or diagnosis and/or treatment in wound healing, and/or its pathological disorders and/or for identification of pharmacologically active substances.

The invention relates to polypeptides or nucleic acids encoding these of a family of G-protein coupled receptors and their use for the diagnosis, prevention and/or treatment of disorders, for example, skin disorders, and/or diagnosis and/or treatment in wound healing, and/or its pathological disorders and for the identification of pharmacologically active substances.

Wounds in general heal without therapeutic intervention. However, there are numerous disorders in which wound healing plays a role, such as, for example, diabetes mellitus, arterial occlusive diseases, psoriasis, Crohn's disease, epidermolysis bullosa, age-related skin changes or innervation disorders. Wound healing disorders lead to a delayed healing of wounds or to chronic wounds. These disorders can be caused by the nature of the wounding (e.g. large-area wounds, deep and mechanically expanded operation wounds, burns, trauma, de-cubitus), medicinal treatment of the patients (e.g. with corticoids) but also by the nature of the underlying disorder itself. For example, 25% of the patients with Type II diabetes thus frequently suffer from chronic ulcers (“diabetic foot”), of which approximately half necessitate expensive in-patient treatments and nevertheless finally heal poorly. Diabetic foot causes more stays in hospital than any other complication associated with diabetes. The number of these cases in diabetes Type I and II is on the increase and represents 2.5% of all hospital admissions. Moreover, wounds heal more poorly with increasing age of the patients. An acceleration of the natural wound healing process is often desirable as well in order to decrease, for example, the danger of bacterial infections or the rest periods of the patients.

Further wound healing disorders can also occur after successful wound closure. While foetal skin wounds heal without scar formation, scars always occur after injuries in the postnatal period , which often represents a great cosmetic problem. In the case of patients with large-area burn wounds, the quality of life can moreover be dramatically adversely affected, especially as in scarred skin the appendages, such as hair follicles, sweat and sebaceous glands are missing. In the case of a genetic disposition, keloids can also occur, hypertrophic scars which proliferate into the surrounding skin.

The process of skin healing requires complex actions and interactions of various cell types which proceed in a coordinated manner. In the wound healing process, the following steps are differentiated: clotting of blood in the area of the wound, the recruitment of inflammatory cells, reepithelialization, the formation of granular tissue and the matrix remodeling. The exact reaction patterns of the cell types involved during the phases of proliferation, migration, matrix synthesis and contraction are, just like the regulation of genes such as, for example, growth factors, receptors and matrix proteins, little known up to now.

Thus until now only a few satisfactory therapies have been developed in order to be able to intervene in wound healing disorders. Established forms of therapy are restricted to physical assistance of wound healing (e.g. dressings, compresses, gels) or the transplantation of skin tissues, cultured skin cells and/or matrix proteins. In recent years, growth factors have been tested for improving wound healing without, however, improving the conventional therapy decisively. The diagnosis of wound healing disorders is also based on not very meaningful optical analysis of the skin, since a deeper understanding of the gene regulation during wound healing was lacking until now.

Not very satisfactory therapies have been developed until now for other disorders of regenerative processes as well. Here too, the knowledge of gene regulation is advantageous for the development of diagnostics and therapies. It has been shown (Finch et al., 1997, Am. J. Pathol. 151: 1619-28; Werner, 1998, Cy-tokine Growth Factor Rev. 9: 153-165) that genes relevant to wound healing also play a crucial role in dermatological disorders which are based on disorders of the regeneration of the skin, and generally in regenerative processes. Thus the growth factor KGF not only plays a crucial role in the regulation of the proliferation and differentiation of keratinocytes during wound healing, but is also an important factor in the hyperproliferation of the keratinocytes in psoriasis.

Novel possibilities for the modulation of wound healing and the treatment of wound healing disorders can open up through the investigation of G-proteins coupled receptors (GPCR) in connection with wound healing. The super-family of GPCR that constitutes the largest known receptor family until now is characterized through 7 highly conserved characteristic sequence motives of 20-30 amino acid length respectively that have a high hydrophobicity (Probst et al., 1992, DNA and Cell Biol., 11: 1-20; Flower, 1999, Biochem. Biophys. Acta, 1422: 207-234). Signal transduction is usually effected through heterotrimeric G-proteins, which in turn regulate second messengers like cAMP, cGMP, diacyl-glycerol or inositol-1,4,5-trisphosphate (Watson and Arkinstall (Hrsg.), 1999, The G-Protein Receptors Facts Book, Academic Press, New York). It is also possible, however, to activate MAP kinases (Lefkowitz, 1998, J. Biol. Chem., 273: 18677-18680). Stimuli effecting GPCRs comprise a broad spectrum that extends from light, smelling substance, neuromodulators to a variety of hormones (Lerner et al., 1993, Ciba Found. Symp., 179: 76-87) which explains the medical importance of GPCRs. It is estimated that more than 50% of all modern pharmaceuticals effect GPCRs (Gudermann et al., 1995 J. Mol. Med. 73: 51-63). Pharmaceuticals identified so far concentrate on, for example, the modulation of the gonadotropin releasing hormone receptors, thereby allowing treatment of prostate and breast carcinomas as well as endometriosis and early onset puberty (Pace et al., 1992, Am. Fam. Physician, 44: 1777-1782). Propanolol, that is an antagonist of the α-adrenergic receptors of the heart, on the other hand is used for treatment of high blood pressure, angina pectoris and psychogenous disorders (Nace and Wood, 1987, Clin. Pharmacokinet. 13: 51-64; Ananth and Lin, 1986 Neuropsychobiology, 15: 20-27). Metaproterenol, that is an antagonist of the β2-adrenergic receptor of the lung, is applied in the dilatation of the bronchia (Hurst, 1973, Ann. Allergy, 31: 460-466).

However agonists or antagonists of GPCRs are rarely used in wound healing processes. One antagonist of the histamin 2 receptor can, for example, be employed to treat ulcer and idiopathic urticaria (Sontag et al., 1984, N. Engl. J. Med., 311: 689-693; Choy and Middleton, 1991, DICP, 25: 609-612). Furthermore “protease activated receptor 3 proteins” (U.S. Pat. No. 5,892,014), nucleotide receptors (WO 98/32429; WO 94/23723), angiotensin receptors (WO 98/33813), the CCR-5 receptor (WO 98/30218) and the thromboxan A2 receptor (U.S. Pat. No. 4,851,413) have been described as potential targets for the modulation of wound healing and/or treatment in wound healing disorders. Antibodies directed against GPCR of the genfamily PF4AR (IL-8) have been associated with diagnosis and treatment of inflammatory conditions of the skin (U.S. Pat. No. 5,776,457) and wound healing (U.S. Pat. No. 6,087,475). However this claim is based only on experimental data obtained from experiments using lung tissue (U.S. Pat. No. 5,776,457) or characterizing the antibody using neutrophil granulocytes (U.S. Pat. No. 6,087,475). U.S. Pat. No. 6,025,154 describes a G-protein chemokin receptor polypeptide and lists among many diseases wound healing, which may be diagnosed or treated using substances interacting with G-protein chemokin receptor polypeptides. The sequences claimed in (U.S. Pat. No. 6,087,475, U.S. Pat. No. 5,776,457, U.S. Pat. No. 6,025,154) display no significant sequence identity with the poly-peptides and nucleic acids according to the instant invention. It is not a promising strategy for a person skilled in the art to infer from the usability of a GPCR of one gene family for diagnosis and treatment of skin disorders or wound healing to the usability of a GPCR of distantly related gene family for diagnosis and treatment of the same diseases, as due to the diversity of GPCRs and the physiological processes controlled by the GPCRs, it is not possible to predict the function of a GPCR merely based on the fact that a GPCR is a member of the superfamiliy of G-protein coupled receptors.

With exception of the olfactoric GPCRs, of which 1000-2000 are present in humans, several hundred members of the GPCR superfamily have been identified as of now (Flower, Biochem. Biophys. Acta, 1999, 1422: 207-234). More than 80 of those receptors belong to the so called “Orphan” receptors, that have not been assigned a ligand as of yet (Marchese et al., 1999, Trends in Pharmacol. Sci. 20: 370-375). Through the identification and analysis of orphan receptors in connection with disorders novel possibilities for the treatment of those disorders open up (Marchese et al., supra), in particular since a multitude of specialized methods to screen for antagonists and agonists of GPCRs are available (Lerner et al., supra; WO 96/41169; U.S. Pat. No. 5,482,835; WO 99/06535; EP 0 939 902; WO 99/66326; WO 98/34948; EP 0 863 214; U.S. Pat. No. 5,882,994; U.S. Pat. No. 5,891,646). WO 99/41364 discloses a method for identifying wound relevant genes based on the use of “healer”-mice. In particular, healer-mice are characterized by a rapid healing of cartilage, but it is doubtful whether healer mice display enhanced wound healing and healing of the skin and thus whether the mice are suited for the identification of genes relevant to wound healing or skin disorders. Moreover, it is not known which genes are mutated ion healer-mice. Tables 3 and 4 (page 44 and 45) list a number of chromosomal loci affected in healer mice. It is therefore questionable whether genes identified are significant for wound healing or skin disorders in human since the differential activity of genes identified using healer mice might merely reflect effects of the mutated genes as opposed to pathological processes of skin diseases or wound healing.

It is therefore the object of the present invention to make available novel polypeptides of the superfamily of G-protein coupled receptors and nucleic acids encoding these which are involved in processes in disorders of mammalian cells, for example, in disorders of skin cells, and/or in wound healing and/or its pathological disorders and whose use decisively improves the diagnosis, prevention and/or treatment and also the identification and development of pharmaceuticals which are effective in connection with these disorders.

In the analysis of gene expression during the wound healing process it was surprisingly possible to identify genes, unknown until present, that are homologous to each other, that form a gene family within the superfamily of G-protein coupled receptors and that show a moderate homology to the orphan receptor Mas (Young et al. 1986, Cell, 45:711-719), to the “dorsal root” GPCR receptors (WO 99/32519) and to the murine ERG9 GPCR (JP 2000023677), i.e. 30-50% sequence identity of polypeptides. Human SW1695 is known from WO200119983 described as “IGS3 GPCR” and from WO200116159 described as “Ant GPCR”. In contrast, murine SW1695 is new and displays a sequence identity to for example murine CanoMan GPCR (WO200116177) of 53% at the level of amino acids over a region of 331 amino acids. The sequence identity of murine SW1695 to IGS3 and Ant respectively is 53% at the level of amino acids over a region of 314 amino acids. The sequence identity of human SW1368 to IGS3 and Ant respectively is 43% at the level of amino acids over a region of 285 amino acids, whereas the identity of murine SW1368 to Ant and IGS3 respectively is 50% at the level of amino acids over a region of 306 amino acids.

The functions of IGS3 and Ant polypeptides and the nucleic acids encoding the polypeptides are not known. With respect to the origin and isolation of the sequence in the case of Ant GPCR there is only mentioning of the organism (human), whereas in the case of IGS3, the nucleic acid sequence has been generated by superpositioning of overlapping genomic sequences of human origin.

The novel polypeptides of these genes are essential for the wound healing process and offer a new therapeutic approach for the treatment of diseases, especially of diseases of skin cells and/or of wound healing and/or its pathological disorders. The polypeptide sequences of the polypeptides according to the invention that are not identifiable in public databases and their cDNAs are listed in the sequence listing (Polypeptide sequences SEQ ID No. 1 to SEQ ID No. 4, cDNA sequences: SEQ ID No. 5 to SEQ ID No. 8). FIG. 5 and FIG. 6 depict a comparison of human and murine polypeptide sequences of SW1368 (SEQ ID No. 1 and 2) and SW1695 (SEQ ID No. 3 and 4).

The object of the invention is achieved by at least one polypeptide according to one of SEQ ID No. 1 to SEQ ID No. 4 or functional variants thereof, and/or nucleic acids encoding the polypeptide or variants thereof, as well as their use for the diagnosis, prevention and/or treatment of disorders, for example, skin disorders, and/or diagnosis and/or treatment in wound healing and/or for identification of pharmacologically active substances.

Generally, the analysis of differentially expressed genes in tissues is affected by markedly more errors in the form of false-positive clones than the analysis of cell culture systems. This problem cannot be circumvented by the use of a defined cell culture system, as existing, simple cell culture systems cannot adequately simulate the complexity of the wound-healing process in the tissue.

The problem exists in particular in the skin, which consists of a multiplicity of different cell types. Moreover, the process of wound healing is a highly complicated process which includes temporal and spatial changes of cellular processes, such as proliferation and differentiation, in the different cell types. The approach to investigate not only the complex cell system skin, but moreover the physiological process of wound healing and even different wound-healing stages at the level of differentially expressed genes is therefore not a promising strategy for a person skilled in the art. On account of these difficulties, the success of the screening was significantly dependent on the choice of the experimental parameters. While the methods used (e.g. subtractive hybridization) are standard methods, the screening and verification strategy is already inventive per se owing to the elaborate and defined choice of parameters. For example, the time of biopsy taking is critical for the success of the screening: wound-healing disorders and skin diseases are often based on disorders in cell proliferation and cell migration. These processes are initiated one day after wounding, which is why analysis of the molecular processes before this time would yield little information about the processes which are essential for normally proceeding wound healing. On the other hand, in the course of wound healing, the composition of the cell types in the wound changes greatly later than one day after wounding. This can lead to a differential expression of a specific gene in the wound being measured which is based not on altered expression in the cells, but only on the different cell composition. This illustrates that the choice of the day of biopsy taking crucially affected the success of the screening. Despite the defined parameters, an overrepresentation of genes was observed, which are differentially expressed during wound healing, but which are unsuitable for use in wound healing or in skin diseases. These genes include, for example, genes which code for enzymes of the primary metabolism, such as glycolysis, citrate cycle, gluconeogenesis and respiratory chain, but also genes which code for ribosomal proteins, e.g. L41 and S20. Only a comparatively small number of genes were identified as suitable. An identification of the GPCR genes according to the invention as genes relevant to wound healing was therefore surprising.

Moreover, there are enormous variabilities in the state of the wound at the time of a possible biopsy of the patient on initial contact with the physician. An animal model was therefore used for the identification of the previously described nucleic acids. BALB/c mice were wounded and wound biopsies were taken at different times. This procedure has the advantage that conditions such as genetic background, nature of the wound, time of the biopsy etc. can be exactly controlled and thereby allow a reproducible analysis of gene expression. Even under the defined mouse conditions, further methodical problems arise such as redundancy of the analyzed clones and underrepresentation of weakly expressed genes, which make the identification of relevant genes difficult.

Generally, the analysis of differentially expressed genes in tissues is affected by markedly more errors in the form of false-positive clones than the analysis of cell culture systems. Since the process of wound healing involves a multiplicity of different cell types, whose composition and gene expression pattern undergoes changes during the whole course of wound healing, the analysis of differentially expressed genes results in a very low number of hits.

In the presented analysis of gene expression SW1368 was identified as being differentially expressed in wounds: SW1368 was enriched in a fraction of a cDNA population that was obtained by subtracting wounds against intact skin (Example 1).

After the primary identification of a gene, it is necessary to confirm wound healing-specific expression by a further method. This was carried out with the aid of “reverse Northern blots” and “TaqMan analysis”. Using these methods, the amount of mRNA in tissues from various wound-healing states of 10 weeks old mice and/or of old and young mice and/or of mice with diabetes was determined. The wound-specific expression of SW1368 in a cDNA library was verified by using “Reverse Nothern Blots” (Example 1, FIG. 1). Moreover, using “TaqMan Assays” the strong expression of SW1368 in normally healing wounds of mice as well as in wounds of old and young mice relative to intact skin was quantified (Example 2, FIG. 2). Furthermore, SW1368 as well as SW1695, which was identified based on sequence homology to SW1368 by means of PCR using degenerate primers, exhibited an altered expression in other wound healing disorders of the mouse (Example 2 and FIG. 3). The expression of SW1368 in mice with diabetes was 50% lower than the expression in wounds of control mice, whereas the level of expression of SW1695 in badly healing wounds of dexamethasone treated mice was three times the level of expression in the wounds of control mice. This shows, that the regulated expression of the genes of the gene-family according to the invention not only plays a role during wound healing but is also necessary for the prevention of pathological course of wound healing. The significance of genes gene family according to the invention could be confirmed by the use of “TaqMan analysis” using human day-1 and day-5 wounds, which exhibited a significant reduction of the level of expression of human SW1695 as well as an up-regulation of the level of expression of human SW1368 during determination of the kinetics of wound healing. None of the two genes was detected as being expressed in the wound ground of ulcers. This shows that the expression of GPCR according to the invention are not only differentially regulated but moreover the dysregulation of expression of these genes can lead to severe wound healing disorders. In addition, human SW1695 was found to be dysregulated in psoriatic skin: a significantly higher expression of the gene was found in non-affected skin biopsies of psoriasis patients compared to lesional, affected skin of the same patients, which evidences that dysregulation of gene expression of these genes can be a diagnostic of and can also cause skin disorders, especially psoriasis.

For the confirmation and generation of full-length cDNA sequences of the nucleic acids described above, full-length clones were generated using colony ony-hybridization (Sambrook et al., 1989, Molecular cloning: A Laboratory Manual, Cold Spring Harbor, Cold Spring Harbor Laboratory Press, New York, Kapitel 8-10) and/or PCR based methods (“RACE”, Frohman et al., 1988, Proc. Natl. Acad. Sci. USA 85: 8998-9002, Chenchik et al., 1996, in A Laboratory Guide to RNA: Isolation, Analysis, and Synthesis, Ed. Krieg, Wiley-Liss, Seiten 272-321; “LDPCR”, Barnes, 1994, Proc. Natl. Acad. Sci. USA 91: 2216-20; ,,“IPCR”, Hartlund Ochman, 1994, Methods Mol. Biol., 31: 187-196) for the mouse-genes as well as for the human genes and the sequences were determined.

The term “functional variants” of a polypeptide within the meaning of the present invention include GPCR polypeptides according to the invention which are regulated, for example, like the polypeptides according to the invention during disease, in particular skin diseases, or in regenerative processes of the skin, but in particular in wound-healing and its disorders. Functional variants, for example, also include polypeptides which are encoded by a nucleic acid which is isolated from non-skin-specific tissue, e.g. embryonic tissue, but after expression in a cell involved in wound healing or skin disease have the designated functions.

Functional variants within the meaning of the present invention are also polypeptides which have a sequence homology, in particular a sequence identity, of about 70%, preferably about 80%, in particular about 90%, especially about 95%, with the polypeptide having the amino acid sequence according to one of SEQ ID No. 1 to SEQ ID No. 4. Examples of such functional variants are accordingly the polypeptides homologous to a polypeptide useable according to the invention, which originate from organisms other than the human or the mouse, preferably from non-human mammals such as, for example monkeys, pigs and rats. Other examples of functional variants are polypeptides which are encoded by different alleles of the gene, in different individuals or in different organs of an organism.

In order to decide, whether a candidate polypeptide is a functional variant, the activity of the candidate functional variant polypeptide may be compared with the activity of a polypeptide according to the invention. Assuming that the candidate functional variant polypeptide fulfills the criteria of a functional variant on the level of % sequence identity the candidate functional variant molecule represents a functional variant if the activity in the functional assays is similar to or identical with the activity exhibited by the polypeptide useable according to the invention.

Such standard wound healing assays comprise for example the application of an expression vector containing a nucleic acid coding for the candidate polypeptide or the application of the candidate polypeptide itself or of an antibody directed against the candidate polypeptide or of an antisense oligonucleotide to wounds. After incubation of, for example an expression vector, the progress of wound healing of wounds that have been injected with different expression vectors containing either the nucleic acid coding for the candidate functional variant polypeptide the expression vector containing the nucleic acid coding for the polypeptide according to the invention is compared. Such assays may also be applied to test the activity of candidate functional variant polypeptides in the case of disorders of wound healing employing for example badly healing wounds of dexamethasone-treated animals. For example, it was demonstrated that application of the polypeptide-variants PDGF-A and PDGF-B on badly healing rabbit wounds resulted in a comparable wound healing response (J. Surg. Res., 2000, 93:230-236). Similar tests can be carried out for skin disorders, for example Psoriasis. In this case, an expression vector containing a nucleic acid coding for the candidate polypeptide or the candidate polypeptide itself or an antibody directed against the candidate polypeptide or an antisense oligonucleotide are applied to for example to human afflicted skin areas transplanted onto SCID mice and the course of the skin disorder, for example the healing, is determined, for example by measuring “PASI-score” in the case of psoriasis.

Variants of the polypeptide can also be parts of the polypeptide according to the invention with at least 6 amino acids length, preferably with at least 8 amino acids length, in particular with at least 12 amino acids length. Also included are deletions of the polypeptides according to the invention, in the range from about 1-30, preferably from about 1-15, in particular from about 1-5 amino acids. For example, the first amino acid methionin can be absent without the function of the polypeptide being significantly altered. Also, posttranslational modifications, for example lipid anchors or phosporyl groups maybe present or absent in variants.

Sequence identity is understood as degree of identity (% identity) of two sequences, that in the case of polypeptides can be determined by means of for example BlastP 2.0.1 and in the case of nucleic acids by means of for example BLASTN 2.014, wherein the Filter is set off and BLOSUM is 62 (Altschul et al., 1997, Nucleic Acids Res., 25:3389-3402). “Sequence homology” is understood as similarity (% positives) of two polypeptide sequences determined by means of for example BlastP 2.0.1 wherein the Filter is set off and BLOSUM is 62 (Altschul et al., 1997, Nucleic Acids Res., 25:3389-3402).

The term “coding nucleic acid” relates to a DNA sequence which codes for an isolatable bioactive polypeptide according to the invention or a precursor. The polypeptide can be encoded by a sequence of full length or any part of the coding sequence as long as the specific, for example receptor-activity is retained.

It is known that small alterations in the sequence of the nucleic acids described above can be present, for example, due to the degeneration of the genetic code, or that untranslated sequences can be attached to the 5′ and/or 3′ end of the nucleic acid without its activity being significantly altered. This invention, therefore, also comprises so-called “variants” of the nucleic acids described above.

Variants are understood as meaning all DNA sequences which are complementary to a DNA sequence, which hybridize with the reference sequence under stringent conditions and have a similar activity to the corresponding polypeptide according to the invention.

“Stringent hybridization conditions” are understood as meaning those conditions in which hybridization takes place at 60° C. in 2.5×SSC buffer, followed by a number of washing steps at 37° C. in a lower buffer concentration, and remains stable.

Variants of the nucleic acids can also be parts of the nucleic acid according to the present invention with at least 8 nucleotides length, preferably with at least 18 nucleotides length, in particular with at least 24 nucleotides length.

The term “pharmacologically active substance” in the sense of the present invention is understood as meaning all those molecules, compounds and/or compositions and substance mixtures which can interact under suitable conditions with the nucleic acids, polypeptides or antibodies or antibody fragments described above, if appropriate together with suitable additives and/or auxiliaries. Possible pharmacologically active substances are simple chemical (organic or inorganic) molecules or compounds, but can also include peptides, proteins or complexes thereof. Examples of pharmacologically active substances are organic molecules that are derived from libraries of compounds that have been analyzed for their pharmacological activity. On account of their interaction, the pharmacologically active substances can influence the function(s) of the nucleic acids, polypeptides or antibodies in vivo or in vitro or alternatively only bind to the nucleic acids, polypeptides or antibodies or antibody fragments described above or enter into other interactions of covalent or non-covalent manner with them.

The invention relates to GPCR polypeptides according to the invention or variants thereof according to the SEQ ID No. 1 to SEQ ID No. 4 and/or nucleic acids coding for these or variants thereof.

The polypeptides described above can furthermore be prepared synthetically. Thus, the entire polypeptide or parts thereof can be synthesized, for example, with the aid of the conventional synthesis (Merrifield technique). Parts of the polypeptides described above are particularly suitable for the obtainment of antisera, with whose aid suitable gene expression banks can be searched in order thus to arrive at further variants, preferably functional variants of the above described polypeptides.

Preferably, the nucleic acids used according to the invention are DNA or RNA, preferably a DNA, in particular a double-stranded DNA. The sequence of the nucleic acids can furthermore be characterized by having at least one intron and/or one polyA sequence. The nucleic acids according to the invention can also be used in the form of their antisense sequence.

For the expression of the gene concerned, in general a double-stranded DNA is preferred, the DNA region coding for the polypeptide being particularly preferred. In the case of eukaryotes this region begins with the first start codon (ATG) lying in a Kozak sequence (Kozak, 1987, Nucleic. Acids Res. 15: 8125-48) up to the next stop codon (TAG, TGA or TAA), which lies in the same reading frame to the ATG. In the case of prokaryotes this region begins with the first AUG (or GUG) after a Shine-Dalgarno sequence and ends with the next stop codon (TAA, TAG or TGA), which lies in the same reading frame to the ATG.

Furthermore the nucleic acid sequences according to the invention can be used for the construction of antisense oligonucleotides (Zheng and Kemeny, 1995, Clin. Exp. Immunol. 100: 380-2; Nellen and Lichtenstein, 1993, Trends Biochem. Sci. 18: 419-23; Stein, 1992, Leukemia 6: 967-74) and/or ribozymes (Amarzguioui, et al. 1998, Cell. Mol. Life Sci. 54: 1175-202; Vaish, et al., 1998, Nucleic Acids Res. 26: 5237-42; Persidis, 1997, Nat. Biotechnol. 15: 921-2; Couture and Stinchcomb, 1996, Trends Genet. 12: 510-5). Using antisense oligonucleotides, the stability of the nucleic acid according to the invention can be decreased and/or the translation of the nucleic acid according to the invention inhibited. Thus, for example, the expression of the corresponding genes in cells can be decreased both in vivo and in vitro. Oligonucleotides can therefore be suitable as therapeutics. This strategy is also suitable, for example, for skin, epidermal and dermal cells, in particular if the antisense oligonucleotides are complexed with liposomes (Smyth et al., 1997, J. Invest. Dermatol. 108: 523-6; White et al., 1999, J. Invest. Dermatol. 112: 699-705; White et al., 1999, J. Invest. Dermatol. 112: 887-92). For use as a probe or as an “antisense” oligonucleotide, a single-stranded DNA or RNA is preferred.

Furthermore, a nucleic acid which has been prepared synthetically can be used for carrying out the invention. Thus, the nucleic acid according to the invention can be synthesized, for example, chemically with the aid of the DNA sequences described in SEQ ID No. 5 to SEQ ID No. 8 and/or with the aid of the protein sequences described in SEQ ID No. 1 to SEQ ID No. 4 with reference to the genetic code, e.g. according to the phosphotriester method (see, for example, Uhlmann, E. & Peyman, A. (1990) Chemical Reviews, 90, 543-584, No. 4).

As a rule, oligonucleotides are rapidly degraded by endo- or exo- nucleases, in particular by DNases and RNases occurring in the cell. It is therefore advantageous to modify the nucleic acid in order to stabilize it against degradation, so that a high concentration of the nucleic acid is maintained in the cell over a long period (Beigelman et al., 1995, Nucleic Acids Res. 23: 3989-94; Dudycz, 1995, WO 95/11910; Macadam et al., 1998, WO 98/37240; Reese et al., 1997, WO 97/29116). Typically, such a stabilization can be obtained by the introduction of one or more internucleotide phosphorus groups or by the introduction of one or more non-phosphorus internucleotides.

Suitable modified intemucleotides are summarized in Uhlmann and Peymann (1990 Chem. Rev. 90, 544) (see also Beigelman et al., 1995 Nucleic Acids Res. 23: 3989-94; Dudycz, 1995, WO 95/11910; Macadam et al., 1998, WO 98/37240; Reese et al., 1997, WO 97/29116). Modified internucleotide phosphate radicals and/or non-phosphorus bridges in a nucleic acid which can be employed in one of the uses according to the invention contain, for example, methyl-phosphonate, phosphorothioate, phosphoramidate, phosphorodithioate, phosphate ester, while non-phosphorus internucleotide analogues, for example, contain siloxane bridges, carbonate bridges, carboxymethyl esters, acetamidate bridges and/or thioether bridges. It is also intended that this modification should improve the shelf life of a pharmaceutical composition which can be employed in one of the uses according to the invention.

In a further embodiment of the nucleic acids according to the invention, the nucleic acids according to the inventions are comprised in a vector, preferably in a “shuttle” vector, phagemid, cosmid, expression vector or vector applicable in gene therapy. Furthermore, the above mentioned nucleic acids can be comprises in “knock-out” gene constructs or expression cassettes. An expression cassette within the meaning of the present invention, comprises at least one promoter or enhancer, at least one translation initiation signal, at least one of the nucleic acids described above, one translation termination signal, one transcription termination signal and one polyadenylation signal for the expression in eukaryotes.

Preferably, the vector applicable in gene therapy contains wound- or skin-specific regulatory sequences which are functionally associated with the nucleic acid according to the invention.

Suitable expression vectors can be prokaryotic or eukaryotic expression vectors. Examples of prokaryotic expression vectors are, for expression in E. coli, e.g. the vectors pGEM or pUC derivatives, examples of eukaryotic expression vectors are for expression in Saccharomyces cerevisiae, e.g. the vectors p426Met25 or p426GAL1 (Mumberg et al. (1994) Nucl. Acids Res., 22, 5767-5768), for expression in insect cells, e.g. Baculovirus vectors such as disclosed in EP-B1-0 127 839 or EP-B1-0 549 721, and for expression in mammalian cells, e.g. the vectors Rc/CMV and Rc/RSV or SV40 vectors, which are all generally obtainable.

In general, the expression vectors also contain promoters suitable for the respective cell, such as, for example, the trp promoter for expression in E. coli (see, for example, EP-B1-0 154 133), the MET 25, GAL 1 or ADH2 promoter for expression in yeast (Russel et al. (1983), J. Biol. Chem. 258, 2674-2682; Mumberg, supra), the Baculovirus polyhedrin promoter, for expression in insect cells (see, for example, EP-B1-0 127 839). For expression in mammalian cells, for example, suitable promoters are those which allow a constitutive, regulatable, tissue-specific, cell-cycle-specific or metabolically specific expression in eukaryotic cells. Regulatable elements according to the present invention are promoters, activator sequences, enhancers, silencers and/or repressor sequences.

Examples of suitable regulatable elements which make possible constitutive expression in eukaryotes are promoters which are recognized by the RNA polymerase III or viral promoters, CMV enhancer, CMV promoter, SV40 promoter or LTR promoters, e.g. from MMTV (mouse mammary tumour virus; Lee et al. (1981) Nature 214, 228-232) and further viral promoter and activator sequences, derived from, for example, HBV, HCV, HSV, HPV, EBV, HTLV or HIV.

Examples of regulatable elements which make possible regulatable expression in eukaryotes are the tetracycline operator in combination with a corresponding repressor (Gossen et al. (1994) Curr. Opin. Biotechnol. 5, 516-20).

Preferably, the expression of the genes relevant for skin disorders and wound healing takes place under the control of tissue-specific promoters, for example, under the control of skin-specific promoters such as, for example, the human K10 promoter (Bailleul et al., 1990. Cell 62: 697-708), the human K14 promoter (Vassar et al., 1989, Proc. Natl. Acad. Sci. USA 86: 1563-67), the bovine cytokeratin IV promoter (Fuchs et al., 1988; The biology of wool and hair (ed. G.E. Rogers, et al.), pp. 287-309, Chapman and Hall, London/New York) are particularly to be preferred.

Further examples of regulatable elements which make tissue-specific expression in eukaryotes possible are promoters or activator sequences from promoters or enhancers of those genes which code for proteins which are only expressed in certain cell types.

Examples of regulatable elements which make cell cycle-specific expression in eukaryotes possible are promoters of the following genes: cdc25A, cdc25B, cdc25C, cyclin A, cyclin E, cdc2, E2F-1 to E2F-5, B-myb or DHFR (Zwicker J. and Müller R. (1997) Trends Genet. 13, 3-6). The use of cell cycle regulated promoters is particularly preferred in cases, in which expression of the polypeptides or nucleic acids according to the invention is to be restricted to proliferating cells.

Examples of regulatable elements which make possible metabolically specific expression in eukaryotes are promoters which are regulated by hypoxia, by glucose deficiency, by phosphate concentration or by heat shock.

An example of an regulatable element which makes possible the keratinocyte-specific expression in the skin, is the FiRE-element (Jaakkola et al., 2000, Gen. Ther., 7: 1640-1647). The FiRE element is a AP-1-driven, FGF-inducible response element of the Syndecan-1 gene (Jaakkola et al., 1998, FASEB J., 12: 959-9).

In order to make possible the introduction of nucleic acids as described above and thus the expression of the polypeptide in a eu- or prokaryotic cell by transfection, transformation or infection, the nucleic acid can be present as a plasmid, as part of a viral or non-viral vector. Suitable viral vectors here are particularly: baculoviruses, vaccinia viruses, adenoviruses, adeno-associated viruses and herpesviruses. Suitable non-viral vectors here are particularly: virosomes, liposomes, cationic lipids, or polylysine-conjugated DNA.

Examples of vectors applicable in gene therapy are virus vectors, for example adenovirus vectors, retroviral vectors or vectors based on replicons of RNA viruses (Lindemann et al., 1997, Mol. Med. 3: 466-76; Springer et al., 1998, Mol. Cell. 2: 549-58, Khromykh, 2000, Curr. Opin. Mol Ther.;2:555-569). Eukaryotic expression vectors are suitable in isolated form for gene therapy use, as naked DNA can penetrate, for example, into skin cells on topical application (Hengge et al., 1996, J. Clin. Invest. 97: 2911-6; Yu et al., 1999, J. Invest. Dermatol. 112: 370-5).

Vectors having gene therapy activity can also be obtained by complexing the nucleic acid with liposomes, since a very high transfection efficiency, for example, of skin cells, can thus be achieved (Alexander and Akhurst, 1995, Hum. Mol. Genet. 4: 2279-85). In the case of lipofection, small unilamellar vesicles are prepared from cationic lipids by ultrasonic treatment of the liposome suspension. The DNA is bound ionically to the surface of the liposomes, namely in such a ratio that a positive net charge remains and the plasmid DNA is complexed to 100% of the liposomes. In addition to the lipid mixtures DOTMA (1,2-dioleyloxypropyl-3-trimethylammonium bromide) and DPOE (dioleoxylphospha-tidylethanolamine) employed by Felgner et al. (1987, supra), meanwhile numerous novel lipid formulations were synthesized and tested for their efficiency in the transfection of various cell lines (Behr et al. 1989, Proc. Natl. Acad. Sci. USA 86: 6982-6986; Felgner et al., 1994, J. Biol. Chem. 269:2550-2561; Gao, X. and Huang, 1991, Biochim. Biophys. Acta 1189:195-203). Examples of the novel lipid formulations are DOTAP N-[1-(2,3-dioleoyloxy)propyl]-N,N,N-trimethylammonium ethyl-sulphate or DOGS (TRANSFECTAM; diocta-decylamidoglycylspermine). Other lipids well suited for transfection in keratinocytes in vivo and in vitro are the cationic lipids Cytofectin GS 2888 (U.S. Pat. No. 5,777,153; Lewis et al., 1996, Proc. Natl. Acad. Sci. USA, 93: 3176-3181). Auxiliaries which increase the transfer of nucleic acids into the cell can be, for example, proteins or peptides which are bound to DNA or synthetic peptide-DNA molecules which make possible the transport of the nucleic acid into the nucleus of the cell (Schwartz et al., 1999, Gene Therapy 6:282; Brandén et al., 1999, Nature Biotech. 17:784). Auxiliaries also include molecules which make possible the release of nucleic acids into the cytoplasm of the cell (Planck et al., 1994, J. Biol. Chem. 269:12918; Kichler et al. (1997) Bioconj. Chem. 8:213) or, for example, liposomes (Uhlmann and Peymann, 1990, supra). Another particularly suitable form of gene therapy vectors can be obtained by applying the above described nucleic acid to gold particles and shooting these into tissue, for example, into the skin, or cells with the aid of the so-called gene gun (Wang et al., 1999, J. Invest. Dermatol. 112: 775-81, Tuting et al., 1998, J. Invest. Dermatol. 111: 183-8).

A further form of a vector having gene therapy activity can be prepared by the introduction of “naked” expression vectors into a biocompatible matrix, for example a collagen matrix. This matrix can, for example, be introduced into wounds in order to transfect the immigrating cells with the expression vector and to express the polypeptides according to the invention in the cells (Goldstein and Banadio, U.S. Pat. No. 5,962,427).

For gene therapy use of the above described nucleic acid, it is also advantageous if the part of the nucleic acid which codes for the polypeptide contains one or more non-coding sequences including intron sequences, preferably between promoter and the start codon of the polypeptide, and/or a polyA sequence, in particular the naturally occurring polyA sequence or an SV40 virus polyA sequence, especially at the 3′ end of the gene, as a stabilization of the mRNA can be achieved thereby (Palmiter et al., 1991, Proc. Natl. Acad. Sci. USA 88:478-482; Jackson, 1993, Cell 74:9-14).

Knock-out gene constructs are known to the person skilled in the art, for example, from the U.S. Pat. No. 5,625,122; U.S. Pat. No. 5,698,765; U.S. Pat. No. 5,583,278 and U.S. Pat. No. 5,750,825.

The present invention further relates to a cell, for example, a skin cell, which contains a nucleic acid according to the invention, or which is transformed using one of the above described vectors, expression cassettes, and/or a knock-out gene constructs. Cells can be either prokaryotic or eukaryotic cells, heterologous or autologous cells, examples of prokaryotic cells are E. coli and examples of eukaryotic cells are skin cells, keratinocytes, fibroblasts or endothelial cells, yeast cells, for Example Saccharomyces cerevisiae or insect cells.

A particularly preferred transformed cell is a transgenic embryonic non-human stem cell, which comprises at least one nucleic acid according to the invention, at least one vector, at least one knock-out gene construct and/or at least one expression cassette as described above. Processes for the transformation of cells and/or stem cells are well known to a person skilled in the art and include, for example, electroporation or microinjection.

The invention further relates to a transgenic non-human mammal whose genome comprises at least one nucleic acid according to the invention, one vector, at least one knock-out gene construct, and/or at least one expression cassette as described above. Transgenic animals in general show a tissue-specifically increased expression of the nucleic acids and/or polypeptides and can be used for the analysis of, for example, wound healing disorders. Thus, for example, an activin A transgenic mouse exhibits improved wound healing (Munz et al., 1999, EMBO J. 18: 5205-15) while a transgenic mouse having a dominantly negative KGF receptor exhibits delayed wound healing (Werner et al., 1994, Science 266: 819-22).

Processes for the preparation of transgenic animals, in particular of transgenic mice, are likewise known to the person skilled in the art from DE 196 25 049 and U.S. Pat. No. 4,736,866; U.S. Pat. No. 5,625,122; U.S. Pat. No. 5,698,765; U.S. Pat. No. 5,583,278 and U.S. Pat. No. 5,750,825 and include transgenic animals which can be produced, for example, by means of direct injection of expression vectors according to the invention into embryos or spermatocytes or by means of the transfection of expression vectors into embryonic stem cells (Polites and Pinkert: DNA Microinjection and Transgenic Animal Production, page 15 to 68 in Pinkert, 1994: Transgenic animal technology: a laboratory handbook, Academic Press, London, UK; Houdebine, 1997, Harwood Academic Publishers, Amsterdam, The Netherlands; Doetschman: Gene Transfer in Embryonic Stem Cells, page 115 to 146 in Pinkert, 1994, supra; Wood: Retrovirus-Mediated Gene Transfer, page 147 to 176 in Pinkert, 1994, supra; Monastersky: Gene Transfer Technology; Alternative Techniques and Applications, page 177 to 220 in Pinkert, 1994, supra).

If the above described nucleic acids are integrated into so-called “targeting” vectors or “knock-out” gene constructs (Pinkert, 1994, supra), it is possible after transfection of embryonic stem cells and homologous recombination, for example, to generate knock-out mice which, in general, as heterozygous mice, show decreased expression of the nucleic acid, while homozygous mice no longer exhibit expression of the nucleic acid. The animals thus produced can also be used for the analysis of, for example, wound healing disorders. Thus, for example, the eNOS (Lee et al., 1999, Am. J. Physiol. 277: H1600-1608), Nf-1 (Atit et al., 1999, J. Invest. Dermatol. 112: 835-42) and osteopontin (Liaw et al., 1998, J. Clin. Invest. 101: 967-71) knock-out mice exhibit impaired wound healing. Here too, a tissue-specific reduction of the expression of wound healing-relevant genes, for example in skin-specific cells using the Cre-loxP system (stat3 knock-out, Sano et al., EMBO J 1999 18: 4657-68), is particularly to be preferred. Transgenic and knock-out cells or animals produced in this way can also be used for the screening and for the identification of pharmacologically active substances and/or vectors applicable in gene therapy.

The invention further relates to a process for preparing a polypeptide according to the invention for use in diagnosis and/or treatment of disorders, for example, skin disorders, and/or in diagnosis and/or treatment in wound healing and/or its pathological disorders or for the identification of pharmacologically active substances in a suitable cell, which is wherein at least one nucleic acids according to the invention is expressed in a suitable cell and, optionally the polypeptide is isolated.

The polypeptide according to the invention is prepared, for example, by expression of the above described nucleic acids in a suitable expression system, as already mentioned above, according to the methods generally known to the person skilled in the art. Suitable cells are, for example, the E. coli strains DHS, HB 101 or BL21, the yeast strain Saccharomyces cerevisiae, the insect cell line Lepidopteran, e.g. from Spodoptera frugiperda, or the animal cells COS, Vero, 293, HaCaT, and HeLa, which are all generally obtainable.

The invention further relates to a process for preparing a fusion protein according to the invention, for example for the diagnosis and/or prevention and/or treatment of disorders, for example, skin disorders, and/or diagnosis and/or treatment in wound healing, and/or its pathological disorders and/or for the identification of pharmacologically active substances in a suitable cell, in which a nucleic acid as described above is used.

Fusion proteins are prepared here which contain the polypeptides described above, the fusion proteins themselves already having the function of a polypeptide of the invention or the specific function being functionally active only after cleavage of the fusion portion. Especially included here are fusion proteins having a proportion of about 1-300, preferably about 1-200, in particular about 1-100, especially about 1-50, foreign amino acids. Examples of such peptide sequences are prokaryotic peptide sequences, which can be derived, for example, from the galactosidase of E.coli. Furthermore, viral peptide sequences, such as, for example, of the bacteriophage M13 can also be used in order thus to produce fusion proteins for the phage display process known to the person skilled in the art. The fusion proteins mentioned above are also embodiments of the present invention.

Further preferred examples of peptide sequences for fusion proteins are peptides, that facilitate easier detection of the fusion proteins, these are, for example, “Green-fluorescent-protein” or variants thereof.

For the purification of the proteins described above (a) further polypeptide(s) (tag) can be attached. Protein tags according to the invention allow, for example, high-affinity absorption to a matrix, stringent washing with suitable buffers without eluting the complex to a noticeable extent and subsequently targeted elution of the absorbed complex. Examples of the protein tags known to the person skilled in the art are a (His)₆ tag, a Myc tag, a FLAG tag, a haemagglutinin tag, glutathione transferase (GST) tag, intein having an affinity chitin-binding tag or maltose-binding protein (MBP) tag. These protein tags can be situated N- or C-terminally and/or internally.

The invention further relates to a process for producing an antibody or antibody fragment, preferably a polyclonal or monoclonal antibody, for example for the diagnosis and/or prevention and/or treatment of disorders, for example, skin disorders, and/or for the diagnosis and/or treatment in wound healing, and/or its pathological disorders or for the identification of pharmacologically active substances,.

The process is carried out according to methods generally known to the person skilled in the art by immunizing a mammal, for example a rabbit, with a nucleic acid according to the invention, or with a polypeptide according to the invention or parts thereof having at least 6 amino acid length, preferably having at least 8 amino acid length, in particular having at least 12 amino acid length, if appropriate in the presence of, for example, Freund's adjuvant and/or aluminium hydroxide gels (see, for example, Diamond et al., 1981, The New England Journal of Medicine, 1344-1349). The polyclonal antibodies formed in the animal as a result of an immunological reaction can then be easily isolated from the blood according to generally known methods and purified, for example, by means of column chromatography. Monoclonal antibodies can be produced, for example, according to the known method of Winter & Milstein (Winter, G. & Milstein, C. (1991) Nature, 349, 293-299).

The present invention further relates to an antibody or antibody fragments directed against a polypeptide described above and reacts specifically with the polypeptides described above, where the above-mentioned parts of the polypeptide are either immunogenic themselves or can be rendered immunogenic by coupling to suitable carriers, such as, for example, bovine serum albumin, or can be increased in their immunogenicity. This antibody is either polyclonal or monoclonal, preferably it is a monoclonal antibody. The term antibody or antibody fragment is understood according to the present invention as also meaning antibodies or antigen-binding parts thereof prepared by genetic engineering and optionally modified, such as, for example, chimeric antibodies, humanized antibodies, multifunctional antibodies, bi- or oligospecific antibodies, single-stranded antibodies, F(ab) or F(ab)₂ fragments (see, for example, EP-B1-0 368 684, U.S. Pat. No. 4,816,567, U.S. Pat. No. 4,816,397, WO 88/01649, WO 93/06213, WO 98/24884). The antibodies according to the invention can for example be used for diagnosis and/or prevention and/or treatment of diseases, for example skin diseases, and/or for diagnosis and/or treatment of wound healing and/or its pathological disorders, and/or for the identification of pharmacologically active substances.

As alternatives to the classical antibodies, for example, “anticalins” based on lipocalin can be used (Beste et al., 1999, Proc. Natl. Acad. Sci. USA, 96:1898-1903). The natural ligand-binding sites of the lipocalins, such as the retinol-binding protein or the bilin-binding protein can be modified, for example, by a “combinatorial protein design” approach in a manner such that they bind to selected haptens, for example to the polypeptides useable according to the invention (Skerra, 2000, Biochim. Biophys. Acta 1482:337-50). Further known “scaffolds” are known as alternatives for antibodies for molecular recognition (Skerra, J. Mol. Recognit., 2000, 13:167-187).

Thus, for example, the local injection of monoclonal antibodies against TGF beta 1 can improve wound healing in the animal model (Ernst et al., 1996, Gut 39: 172-5).

The invention furthermore relates to using at least one polypeptide according to the invention, and/or fusion protein according to the invention, and/or at least one nucleic acid according to the invention and/or at least one antibody or an antibody fragment according to the invention and/or at least one cell according to the invention, for production of a pharmaceutical preparation for diagnosis and/or prevention and/or treatment of skin disorders, and/or for diagnosis and/or treatment of wound healing and/or its pathological disorders. A pharmaceutical preparation in the sense of the invention encompasses pharmaceuticals, i.e. medicaments, and diagnostics which can be used for diagnosing, preventing or treating a disorder.

The present invention also relates to a process producing a pharmaceutical for the treatment and/or prevention of disorders, for example, skin disorders, and/or treatment in wound healing and/or its pathological disorders, in which at least one nucleic acid according to the invention, at least one polypeptide according to the invention or at least one antibody according to the invention, or at least one cell according to the invention is combined with suitable additives and auxiliaries.

The present invention furthermore relates to a pharmaceutical produced by this process for the treatment and/or prevention of disorders, for example, skin disorders, and/or treatment and/or prevention of wound healing and/or its pathological disorders, which contains at least one nucleic acid, at least one polypeptide or at least one antibody or antibody fragment or at least a cell expressing a polypeptide according to the invention or a nucleic coding for the polypeptide, as described above, if appropriate together with suitable additives and auxiliaries. The invention furthermore relates to the use of this pharmaceutical for the treatment of disorders, for example, skin disorders, and/or treatment in wound healing and/or its pathological disorders.

For gene therapy use in skin disorders, and/or in wound healing, for example in disordered wound healing in human, an especially suitable pharmaceutical is one which contains the described nucleic acid in naked form or in the form of one of the vectors applicable in gene therapy described above or in a form complexed with liposomes or gold particles. The pharmaceutical carrier is, for example, a physiological buffer solution, preferably having a pH of about 6.0-8.0, preferably of about 6.8-7.8, in particular of about 7.4, and/or an osmolarity of about 200-400 milliosmol/liter, preferably of about 290-310 milliosmol/liter. In addition, the pharmaceutical carrier can contain suitable stabilizers, such as nuclease inhibitors, preferably complexing agents such as EDTA and/or other auxiliaries known to the person skilled in the art. The nucleic acid described is optionally administered in the form of the virus vectors described above in greater detail or as liposome complexes or a gold particle complex, commonly topically and locally in the area of the wound. It is also possible to administer the polypeptide itself with suitable additives and/or auxiliaries, such as physiological saline solution, demineralized water, stabilizers, protease inhibitors, gel formulations, such as white petroleum jelly, highly liquid paraffin and/or yellow wax, etc., in order to affect wound healing immediately and directly.

The therapy of the disorders, for example, skin disorders, and/or treatment in wound healing and/or its pathological disorders can be carried out by means of the pharmaceuticals according to the invention through oral dosage forms, such as, for example, tablets or capsules, via the mucous membranes, for example, the nose or the oral cavity, or in the form of dispositories implanted under the skin. Transdermal therapeutic systems (TTS) are known for example, from EP0944398A1, EP0916336A1, EP0889723A1 or EP0852493A1.

Therapy can also be carried out in a conventional manner, e.g. by means of dressings, plasters, compresses or gels which contain the pharmaceuticals according to the invention. This therapy is, for example, preferred for the therapy of skin disorders and/or in wound healing. It is thus possible to administer the pharmaceuticals containing the suitable additives or auxiliaries, such as, for example, physiological saline solution, demineralized water, stabilizers, proteinase inhibitors, gel formulations, such as, for example, white petroleum jelly, highly liquid paraffin and/or yellow wax, etc., topically and locally in order to influence wound healing immediately and directly. The administration of the pharmaceuticals according to the invention can furthermore also be carried out topically and locally in the area of the wound, if appropriate in the form of liposome complexes or gold particle complexes. Furthermore, the treatment can be carried out by means of a TTS, which makes possible a temporally controlled release of the pharmaceuticals according to the invention. A therapy based on the use of cells, which express at least one of the polypeptides according to the invention, of functional variants thereof or nucleic acids coding for the polypeptide or variants thereof can be achieved by using autologous or heterologous cells. Preferred cells comprise skin cells, for example dermal or epidermal cells, especially keratinocytes, fibroblasts and endothelial cells. The cells can be applied to the tissue, preferably to skin, especially preferred to skin wounds directly or together with suitable carrier material (U.S. Pat. No. 5,980,888; WO 92/06179; EP 0242 270; WO 90/02796).

Examples of disorders of skin cells and skin disorders within the meaning of the invention is understood as psoriasis, eczema, especially atopic eczema, acne, urticaria, disorders of pigmentation of the skin, especially vitiligo, senile skin and disorders of hair growth and hair metabolism.

Wound healing within the meaning of the invention is understood as the healing process of a mechanical wound of the skin, such as for example laceration, skin abrasion or excoriation of the skin, for example by means of a permanent load, for example decubitus or necrotic processes, for example Necrobiosis lipoidica.

Examples of pathological wound healing disorders in the meaning of the invention comprise wounds of patients suffering from diabetes or alcoholism, wounds infected with organisms or viruses, ischemic wounds, wounds of patients suffering from arterial disorders, or venous insufficiency, and scars, preferably overshooting scars, especially keloids. Especially preferred badly healing wounds comprise diabetic, neuropathic, venous or arterial and decubitus ulcers, especially venous ulcers.

The present invention furthermore relates to a process for preparing a diagnostic for the diagnosis of disorders, for example, skin disorders, and/or diagnosis in wound healing, and/or its pathological disorders, wherein at least one nucleic acid, at least one polypeptide or at least one antibody or at least one cell expressing at least one polypeptide according to the invention or a nucleic acid coding for the polypeptide, as described above is used, if appropriate is combined with suitable additives and auxiliaries.

For example, it is possible according to the present invention to prepare a diagnostic based on the polymerase chain reaction (Examples 2-6, PCR diagnostic, e.g. according to EP 0 200 362) or an RNase protection assay (see, for instance, Sambrook et al., supra chapter 7, page 7.71-7.,78, Werner et al., 1992, Growth Factor and Receptors: A Practical Approach 175-197, Werner, 1998, Proc. Natl. Acad. Sci. U.S.A. 89: 6896-699) with the aid of a nucleic acid as described above. These tests are based on the specific hybridization of a nucleic acids with its complementary counter strand, usually of the corresponding MRNA or its cDNA. The nucleic acid described above can in this case also be modified, such as disclosed, for example, in EP 0 063 879. Preferably a DNA fragment is labelled according to generally known methods by means of suitable reagents, e.g. radioactively with α-³²P-dCTP or non-radioactively with biotin or digoxigenin, and incubated with isolated RNA, which has preferably been bound beforehand to suitable membranes of, for example, nitrocellulose or nylon. With the same amount of investigated RNA from each tissue sample, the amount of mRNA which was specifically labelled by the probe can thus be determined. Alternatively, the determination of mRNA amount can also be carried directly out in tissue sections with the aid of in situ hybridization (Werner et al., 1992, Proc. Natl. Acad. Sci. USA 89: 6896-6900).

With the aid of the diagnostic according to the invention, can thus also be specifically measured in a tissue sample the strength of expression in order to be able to safely diagnose, for example, a wound healing disorder or dermatological disorders (Examples 2 to 6). Such a process is particularly suitable for the early prognosis of disorders.

The present invention furthermore relates to a diagnostic for the diagnosis of disorders, for example, skin disorders and/or for the diagnosis in wound healing and/or its pathological disorders, which comprises at least one nucleic acid, at least one polypeptide, at least a cell expressing a polypeptide according to the invention or a nucleic acid coding for the polypeptide or at least one antibody, as described above, if appropriate together with suitable additives and auxiliaries.

A preferred diagnostic according to the invention contains the described polypeptide or the immunogenic parts thereof described in greater detail above. The polypeptide or the parts thereof, which are preferably bound to a solid phase, e.g. of nitrocellulose or nylon, can be brought into contact in vitro, for example, with the body fluid to be investigated, e.g. wound secretion, in order thus to be able to react, for example, with autoimmune antibodies. The antibody-peptide complex can then be detected, for example, with the aid of labelled anti-human IgG or antihuman IgM antibodies. The labeling involves, for example, an enzyme, such as peroxidase, which catalyses a color reaction. The presence and the amount of autoimmune antibody present can thus be detected easily and rapidly by means of the color reaction.

A further diagnostic, that is that subject matter of the present invention, contains the antibodies according to the invention themselves. With the aid of these antibodies, it is possible, for example, to easily and rapidly investigate a tissue sample as to whether the concerned polypeptide is present in an increased amount in order to thereby obtain an indication of possible disorders, for example, skin disorders and wound healing disorders. In this case, the antibodies according to the invention are labelled, for example, with an enzyme, as already described above. The specific antibody-peptide complex can thereby be detected easily and rapidly by means of an enzymatic color reaction.

A further diagnostic according to the invention comprises a probe, preferably a DNA probe, and/or primer. This opens up a further possibility of obtaining the described nucleic acids, for example by isolation from a suitable gene bank, for example from a wound-specific or skin specific or skin disorder-specific gene bank, with the aid of a suitable probe (see, for example, J. Sambrook et al., 1989, Molecular Cloning. A Laboratory Manual 2nd edn., Cold Spring Harbor Laboratory, Cold Spring Harbor, NY Chapter 8 page 8.1 to 8.81, Chapter 9 page 9.47 to 9.58 and Chapter 10 page 10.1 to 10.67).

Suitable probes are, for example, DNA or RNA fragments having a length of about 100-1000 nucleotides, preferably having a length of about 200-500 nucleotides, in particular having a length of about 300-400 nucleotides, whose sequence can be derived from the polypeptides according to SEQ ID No. 1 to SEQ ID No. 4 of the sequence protocol and/or with the aid of the cDNA sequences, that are indicated in the sequence protocol according to SEQ ID No. 5 to SEQ ID No. 8.

Alternatively, it is possible with the aid of the derived nucleic acid sequences to synthesize oligonucleotides which are suitable as primers for a polymerase chain reaction. Using this, the nucleic acid described above or parts of this can be amplified and isolated from cDNA, for example wound-specific cDNA (Examples 2 to 6) or psoriasis-specific cDNA (Example 7). Suitable primers are, for example, DNA fragments having a length of about 10 to 100 nucleotides, preferably having a length of about 15 to 50 nucleotides, in particular having a length of 20 to 30 nucleotides, whose sequence can be derived from the polypeptides according to SEQ ID No. 1 to SEQ ID No. 4 of the sequence listing and/or with the aid of the CDNA sequences, that are indicated in the sequence listing according to SEQ ID No. 5 to SEQ ID No. 8.

A further subject matter of the invention relates to the use of diagnostic according to the invention for diagnosis of disorders, for example, skin disorders, and/or diagnosis in wound healing and/or its pathological disorders.

The invention furthermore relates to a process for preparing a test for the discovery of for example pharmacologically active substances, wherein at least one nucleic acid according to the invention, at least one polypeptide according to the invention, at least one fusion protein according to the invention, at least one antibody or antibody fragment according to the invention or at least one cell according to the invention, if appropriate together with suitable additives and auxiliaries, is used for preparing the test.

The invention furthermore comprises a test produced according to the invention for the identification of pharmacologically active substances which comprises at least one nucleic acid according to the invention, at least one polypeptide according to the invention or at least one antibody according to the invention, at least one cell according to the invention, if appropriate together with suitable additives and auxiliaries. Such tests can be employed for the identification of pharmacologically active substances in connection with diseases, preferably skin diseases, wound healing and/or its pathological disorders.

In a preferred embodiment of the invention, at least one cell expressing at least one GPCR polypeptide according to the invention or a functional variant thereof or a nucleic acid coding for these or a variant thereof is used for the test according to the invention.

The invention furthermore comprises a test, wherein at least one GPCR polypeptide according to the invention and/or at least one nucleic acid according to the invention and/or at least one antibody or an antibody fragment according to the invention, is bound to a solid-phase. Such tests can be employed for the identification of pharmacologically active substances in connection with diseases, preferably skin diseases, wound healing and/or its pathological disorders.

A suitable system can be produced, for example, by the stable transformation of cells, for example, epidermal or dermal cells with expression vectors which contain selectable marker genes and the described nucleic acids. In this process, the expression of the described nucleic acids is altered in the cells such that it corresponds to the pathologically disturbed expression in vivo. Anti-sense oligonucleotides which contain the described nucleic acid can also be employed for this purpose. It is therefore of particular advantage for these systems to know the expression behavior of the genes in disturbed regenerative processes, such as disclosed in this application. Often, the pathological behavior of the cells in vitro can thus be imitated and substances can be sought which reproduce the normal behavior of the cells and which have a therapeutic potential.

Suitable cells for these test systems are, for example, HaCaT cells, which are generally obtainable, and the expression vector pCMV4 (Anderson et al., 1989, J. Biol. Chem. 264: 8222-9). The nucleic acid as described above can in this case be integrated into the expression vectors both in the sense and in the anti-sense orientation, such that the functional concentration of mRNA of the corresponding genes in the cells is either increased, or is decreased by hybridization with the antisense RNA. After the transformation and selection of stable transformants, the cells in culture in general show an altered proliferation, migration and/or differentiation behavior in comparison with control cells. This behavior in vitro is often correlated with the function of the corresponding genes in regenerative processes in the body (Yu et al., 1997, Arch. Dermatol. Res. 289: 352-9; Mils et al., 1997, Oncogene 14: 15555-61; Charvat et al., 1998, Exp Dermatol 7: 184-90; Werner, 1998, Cytokine Growth Factor Rev. 9: 153-65; Mythily et al., 1999, J. Gen. Virol. 80: 1707-13;) and can be detected using tests which are simple and rapid to carry out, such that test systems for pharmacologically active substances based thereon can be constructed. Thus, the proliferation behavior of cells can be detected very rapidly by, for example, the incorporation of labelled nucleotides into the DNA of the cells (see, for example, Savino and Dardenne, 1985, J. Immunol. Methods 85: 221-6; Perros and Weightman, 1991, Cell Prolif. 24: 517-23; de Fries and Mitsuhashi, 1995, J. Clin. Lab. Anal. 9: 89-95), by staining the cells with specific stains (Schulz et al., 1994, J. Immunol. Methods 167: 1-13) or by means of immunological processes (Frahm et al., 1998, J. Immunol. Methods 211: 43-50). The migration can be detected simply by the migration index test (Charvat et al., supra) and comparable test systems (Benestad et al., 1987, Cell Tissue Kinet. 20: 109-19, Junger et al., 1993, J. Immunol. Methods 160: 73-9). Suitable differentiation markers are, for example, keratin 6, 10 and 14 and also loricrin and involucrin (Rosenthal et al., 1992, J. Invest. Dermatol. 98: 343-50), whose expression can be easily detected, for example, by means of generally obtainable antibodies.

Another suitable test system is based on the identification of interactions using the so-called two-hybrid system (Fields and Sternglanz, 1994, Trends in Genetics, 10, 286-292; Colas and Brent, 1998 TIBTECH, 16, 355-363). In this test, cells are transformed using expression vectors which express fusion proteins from the polypeptide described above and a DNA binding domain of a transcription factor such as, for example, Gal4 or LexA. The transformed cells additionally contain a reporter gene, whose promoter contains binding sites for the corresponding DNA binding domains. By transformation of a further expression vector which expresses a second fusion protein from a known or unknown polypeptide having an activation domain, for example of Gal4 or Herpes simplex virus VP16, the expression of the reporter gene can be greatly increased if the second fusion protein interacts with the polypeptide described above. This increase in expression can be utilized in order to identify novel pharmacologically active substances, for example by preparing a cDNA library from regenerating tissue for the construction of the second fusion protein. Moreover, this test system can be utilized for the screening of substances which inhibit an interaction between the polypeptide described above and pharmacologically active substance. Such substances decrease the expression of the reporter gene in cells which express fusion proteins of the polypeptide described above and of the pharmacologically active substance (Vidal and Endoh, 1999, Trends in Biotechnology; 17: 374-81). Novel active compounds which can be employed for the therapy of disorders of, for example, regenerative processes can thus be rapidly identified.

Additional cell-based test systems suitable for the analysis of GPCR polypeptides are summarized in Marchese et al. (1999, Trends in Pharmacol. Sci. 20: 370-375) and comprise so-called “ligand screening assays” especially suitable for cells with a low endogeneous GPCR background expression or with a well measurable level of expression of G-proteins or both. For example in yeast cells the pheromon receptor can be replaced by a GPCR according to the invention. The effect of test substances on the receptor can be determined upon modulation of histidine synthesis, i.e. by growing in histidine-free medium. In addition using cells transfected with nucleic acids according to the invention it can be analyzed whether test substances mediate translocation of a detectable arrestins, for example of a arrestin-GFP-fusion protein. Moreover, it can be analyzed whether test substances mediate GPCR-mediated dispersion or aggregation of Xenopus laevis melanophores. Another test system utilizes the universal adapter G-protein G alphal6, which mobilizes Ca²⁺. Other screening test systems are described in Lemer et al., supra; WO96/41169; U.S. Pat. No. 5,482,835; WO99/06535; EP 0 939 902; WO99/66326; WO98/34948; EP 0 863 214; U.S. Pat. No. 5,882,944 and U.S. Pat. No. 5,891,641.

A further preferred embodiment of the invention is a test for the identification of pharmacologically active substances, wherein pharmacologically active substances are tested for whether they exert an effect onto a transgenic animal according to the invention in connection with disorders, preferably skin disorders or wound healing or its pathological disorders. Transgenic animals can be tested for example for altered wound healing properties, or altered epidermal proliferation, in particular stimulated epidermal proliferation or altered inflammation. Candidate pharmacological substances can then be tested, whether, for example, they alter the wound healing properties, for example stimulate wound healing or whether they alter, for example, the epidermal proliferation or inflammation.

A further preferred embodiment of the invention is a test for the identification of pharmacologically active substances, wherein pharmacologically active substances are tested for whether they exert an effect onto the expression of at least one nucleic acid according to the invention.

Assays for the identification of pharmacologically active substances that influence the expression of genes are known to the person skilled in the art (for example Sivaraja et al., 2001, U.S. Pat. No. 6,183,956).

Therefore cells that express nucleic acids according to the invention, for example HeLa cells, can be cultivated as a test system for the analysis of gene expression in vitro, wherein skin cells, especially keratinocytes, fibroblasts or endothelial cells are preferred. A possible test system is the human keratinocyte cell line HaCaT which is generally available.

The analysis of gene expression takes place for example at the level of MRNA or proteins. The amount of nucleic acid according to the invention or of protein is measured upon addition of one or more substances to the cell culture and compared with the appropriate amount in a control culture. This is done using, for example by means of hybridizing an anti-sense probe, with the mRNA of target genes according to the invention contained in the lysate of cells. The hybridization can be quantified by binding a specific antibody to the mRNA-probe complex (see Stuart and Frank, 1998, U.S. Pat. No. 4, 732,847). It is possible to undertake the analysis in high-throughput scale and test many substances for their suitability as a modulator of expression of nucleic acids according to the invention (Sivaraja et al., 2001, U.S. Pat. No. 6,183,956). The substances to be analyzed can for example be derived from libraries of substances (see for example DE 19816414, DE 19619373) containing thousands of very heterogeneous substances. As an alternative the RNA or mRNA can be isolated from the cells and subsequently the absolute amount or the relative amount of mRNA of target genes according to the invention can be quantified by means of quantitative RT-PCR (see EP 0 200 362; Wittwer et al., 1997, BioTechniques 22:130-138; Morrison et al., 1998, BioTechniques 24:954-962) or by means of RNAse protection assays (see for example Sambrook et al., 1989, Molecular Cloning: A Laboratory Manual, Cold Spring Harbor, Cold Spring Harbor Laboratory Press, New York, Chapter 7; EP 0 063 879). Another possibility is to analyze the amount of protein in the cell lysate using antibodies recognizing polypeptide according to the invention. In this case quantification is established by means of a ELISA or a Western-Blot, generally known to the person skilled in the art. In order to determine the specificity of the effect of the substances onto the expression of nucleic acids according to the invention the influence of substances onto the expression of target genes is compared with the influence of the substances onto the expression of other genes such as for example genes of the metabolism, e.g. GAPDH. Such analysis can be performed in separate analysis or in parallel to the analysis of nucleic acids according to the invention.

In another test system the polypeptides according to the invention or functional variants thereof and/or nucleic acids coding for the polypeptides or variants thereof, and/or antibodies or antibody fragments directed against polypeptides according to the invention or functional variants thereof and/or cells expressing polypeptides according to the invention or functional variants thereof or nucleic acids coding for these or variants thereof are bound to a solid phase and substances are tested for interaction for example for binding or change of conformation. Suitable systems such as affinity chromatography and fluorescence spectroscopy are known to the person skilled in the art.

The solid-phase bound polypeptides according to the invention or functional variants thereof or nucleic acids coding for the polypeptides or variants thereof, fusion proteins according to the invention or antibodies or antibody fragments directed against polypeptides according to the invention or functional variants thereof or cells expressing polypeptides according to the invention or functional variants thereof or nucleic acids coding for these or variants thereof can also be part of an array. Such arrays can be employed for analysis and/or diagnosis of diseases, preferably of skin diseases, wound healing and/or disorders of wound healing.

The invention furthermore comprises an array, wherein at least one GPCR polypeptide according to the invention, and/or at least one fusion protein according to the invention, and/or at least one nucleic acid coding for these, and/or at least one antibody or antibody fragment according to the invention, and/or at least one cell expressing a polypeptide according to the invention or a functional variant thereof or a nucleic acid coding for this or a variant thereof is fixated to a carrier material. Such arrays can be employed for the analysis in connection with disorders of skin cells and/or with wound healing and/or its pathological disorders.

The invention further relates to a method of producing an array fixated to a carrier material, wherein at least one GPCR polypeptide according to the invention and/or at least one fusion protein according to the invention and/or at least one nucleic acid encoding the polypeptide and/or at least one antibody or an antibody fragment directed against the polypeptide, is fixated to said carrier material.

Methods of producing such arrays, for example based on solid-phase chemistry and photo-labile protective groups are generally known (U.S. Pat. No. 5,744,305). Such arrays can also brought into contact with substances or a substance libraries and tested for interaction, for example for binding or change of conformation.

Thus it is possible that a substance to be tested contains a detectable marker, for example the substance can be labeled radioactively, fluorescence labeled or luminescence labeled. Moreover, substances can be coupled to proteins allowing an indirect detection, for example by enzymatic catalysis, by means of a peroxidase-assay with a chromogenic substrate or by means of binding a detectable antibody. Upon interaction with a test substance, changes of conformation of a polypeptide according to the invention can be determined for example by changes of fluorescence of an endogenous tryptophan-residue in the polypeptide.

The pharmacologically active substances that have been identified by means of the test systems can, if appropriate, can be combined all together with suitable additives and/or auxiliaries, for the production of a diagnostic or pharmaceutical for the diagnosis, prevention and/or treatment of diseases of skin cells, and/or for diagnosis and/or treatment in wound healing and/or its pathological disorders.

Pharmacologically active substances of the polypeptides described above can also be nucleic acids which are isolated by means of selection processes, such as, for example, SELEX (see Jayasena, 1999, Clin. Chem. 45: 1628-50; Klug and Famulok, 1994, M. Mol. Biol. Rep. 20: 97-107; Toole et al., 1996, U.S. Pat. No. 5,582,981). In the SELEX process, typically those molecules which bind to a polypeptide with high affinity (aptamers) are isolated by repeated amplification and selection from a large pool of different, single-stranded RNA molecules. Aptamers can also be synthesized and selected in their enantiomorphic form, for example Example as the L-ribonucleotide (Nolte et al., 1996, Nat. Biotechnol. 14: 1116-9; Klussmann et al., 1996, Nat. Biotechnol. 14: 1112-5). Thus isolated forms have the advantage that they are not degraded by naturally occurring ribonucleases and therefore have greater stability.

The identified pharmacologically active substances can, if appropriate, be combined or together with suitable additives and/or auxiliaries for the production of a diagnostic or a pharmaceutical for the prevention, treatment and/or diagnosis of diseases, for example diseases of skin cells and/or for diagnosis and/or treatment of wound healing and/or its pathological disorders. The pharmacologically active substances can be, for example anorganic or organic molecules, for example nucleic acids or analoga of nucleic acids, peptides or proteins. especially antibodies, functional variants of polypeptides according to the invention or nucleic acids encoding these or ligands of polypeptides according to the invention. Examples of pharmacologically active substances are furthermore organic molecules, contained in substance libraries that have been tested for their pharmacological activity.

The use of nucleic acids as a diagnostic can for example be based on the polymerase chain reaction as described above. The use of nucleic acids as pharmaceuticals can be achieved for example gene therapeutically using a vector applicable in gene therapy or by means of application of antisense nucleotides as described above.

The use of other organic or anorganic pharmacologically active substances as pharmaceuticals can result from application as described above. The use of antibodies as diagnostic can result from immunological techniques as described above, for example using antibodies that have been labeled with an enzyme. The specific antibody-peptide complex can be detected easily and rapidly by means of an enzymatic color reaction.

In order to use pharmacologically active substances as a diagnostic, the substances can contain a detectable marker, for example a substance may be radioactively-labeled, fluorescence-labeled or luminescence-labeled. Furthermore, substances may be coupled to enzymes, allowing an indirect detection, for example by enzymatic catalysis by means of a peroxidase-assay using a chromogenic substrate or by binding a labeled or detectable antibody. The substances can be brought into contact with a probe to be analyzed and thus the amount of polypeptide according to the invention, or a nucleic acid according to the invention, or of a cell according to the invention, or an antibody or an antibody fragment according to the invention, can be determined in the probe. The result of the analysis of the probe obtained from an organism can be compared with the results of an analysis of a probe derived from a healthy or pathological organism.

The invention further relates to a process for preparing an array immobilized on a support material for analysis in connection with disorders, for example, skin disorders, and/or in connection with wound healing, in which at least one nucleic acid, at least one polypeptide or at least one antibody or antibody fragment as described above is used for preparation.

Processes for preparing such arrays are known, for example, from U.S. Pat. No. 5,744,305 by means of spotting, printing or solid-phase chemistry in connection with photolabile protective groups.

The invention further relates to an array fixated on a carrier material which comprises at least one nucleic acid, and/or at least one polypeptide, and/or at least one antibody or antibody fragment and/or at least one cell expressing a polypeptide according to the invention or a functional variant thereof or a nucleic acid coding for this or a variant thereof as described above. Such arrays can be used for analysis in connection with disorders, for example, skin disorders and/or in connection with wound healing and/or its pathological disorders.

The invention will now be further illustrated below with the aid of the Figures and examples, without the invention being restricted hereto.

### DESCRIPTION OF THE TABLES FIGURES AND SEQUENCES

FIG. 1: Autoradiograms of hybridizations of membranes with an identical pattern of applied cDNA fragments using two different probes. The cDNA fragments were derived from a wound specific, subtractive cDNA library, that was enriched for those cDNAs, that were stronger expressed in normally healing wounds in comparison to intact skin. Both probes were prepared from cDNAs which originated from subtractive hybridizations. A: skin-specific probe (subtraction intact skin versus normally healing wound), B: wound specific probe (subtraction normally healing wound versus intact skin). The positions of the SW1368 cDNAs (each loaded twice) are indicated with arrows.

FIG. 2: Tabulation of the changed expression of the murine SW1368 and SW1695 genes, that are relevant for wound healing, in normally healing wounds and in poorly healing wounds of 10 weeks old BALB/c mice, that were treated with dexamethasone as well as in wounds of young (4 weeks of age) and old (12 months) BALB/c mice.

FIG. 3: Tabulation of the changed expression of the murine SW1368 and SW1695 genes, that are relevant for wound healing in mice with diabetes and in control animals.

FIG. 4: Tabular survey of the identified polypeptide sequences of the gene family identified in the analysis of gene expression during wound healing and their cDNAs.

FIG. 5: Comparison of the polypeptide sequences of the identified proteins of SW1368 from mouse and human. Exact matches of the mouse sequence of SW1368 with the human sequence of SW1368 are indicated.

FIG. 6: Comparison of the polypeptide sequences of the identified proteins of SW1695 from mouse and human. Exact matches of the mouse sequence of SW1695 with the human sequence of SW1695 are indicated.

FIG. 7: Kinetics of expression of SW1368 during wound healing in human.

FIG. 8: Analysis of the relative level of expression of human SW1368 and SW1695 in the wound ground and wound edge relative to intact skin of ulcus venosum patients.

SEQ ID No. 1 to SEQ ID No. 8 show the polypeptide or cDNA sequences according to the invention from human or mouse.

SEQ ID No. 9 to SEQ ID No. 14 and SEQ ID No. 16 to SEQ ID No. 21 show DNA sequences of oligonucleotides which were used for the experiments of the present invention.

SEQ ID No. 15 shows the part of the sequence of the cDNA used according to the invention according to SEQ ID No. 1, that was determined through sequencing of a clone, that was identified to be regulated by “Reverse Northern Blot analysis”.

### EXAMPLES

### Example 1

### Enrichment of Wound-Relevant cDNA by Means of Subtractive Hybridization and Identification of SW1368 as Wound-Relevant Gene

Total RNA was isolated from intact skin and from wound tissue (wounding on the back 1 day before tissue sampling by scissors cut) of BALB/c mice by standard methods (Chomczynski and Sacchi, 1987, Anal. Biochem. 162: 156-159, Chomczynski and Mackey, 1995, Anal. Biochem. 225: 163-164). The RNAs were then transcribed into cDNA with the aid of a reverse transcriptase. The cDNA synthesis was carried out using the “SMART PCR cDNA synthesis kit” from Clontech Laboratories GmbH, Heidelberg, according to the manual of the manufacturer.

In order to identify those cDNAs which occurred with differing frequency in the cDNA pools, a subtractive hybridization (Diatchenko et al., 1996, Proc. Natl. Acad. Sci. U.S.A. 93: 6025-30) was carried out. This was effected using the “PCR-Select cDNA subtraction kit” from Clontech Laboratories GmbH, Heidelberg, according to the manual of the manufacturer, the removal of excess oligonucleotides after the cDNA synthesis being carried out by means of agarose gel electrophoresis. Two cDNA pools were set up, which were enriched for wound-relevant genes, where one pool was enriched for cDNA fragments which are expressed more strongly in normally healing wound tissue in comparison with intact skin (“wound-specific cDNA pool”), while the other pool was enriched in cDNA fragments which are more strongly expressed in intact skin in comparison with normally healing wound tissue.

In order to identify those genes which were contained in the cDNA pools relevant to wound healing, the presence of the corresponding cDNAs in the pools was analyzed in “reverse Northern blot”. Here, the cDNA fragments are immobilized on membranes in the form of arrays of many different cDNAs, and hybridized with a complex mixture of radio-labeled cDNA (Sambrook et al., 1989, Molecular Cloning: A Laboratory Manual, Cold Spring Harbor, Cold Spring Harbor Laboratory Press, New York, Chapter 9 page 9.47 to 9.58 and Chapter 10 page 10.38 to 10.50; Anderson and Young: Quantitative filter hybridisation; sation; in: Nucleic Acids Hybridization, A Practical Approach, 1985, Eds. Hames and Higgins, IRL Press Ltd.; Oxford, Chapter 4, page 73 to 112).

cDNA fragments were fixed on the membranes used in this example, that were derived from a wound-specific, subtractive cDNA library, that was enriched for those cDNAs, that were stronger expressed in normally healing wounds in comparison with intact skin.

For the preparation of suitable hybridization probes, the subtracted cDNA pools were treated with the restriction endonuclease RsaI and purified by means of agarose gel electrophoresis (Sambrook et al., supra, Chapter 6, page 6.1 to 6.35) in order to separate the cDNA synthesis and amplification primer (see manual “PCR-Select cDNA Subtraction Kit”, Clontech). The cDNAs were then radio-labeled using the “random hexamer priming” method (Feinberg and Vogelstein, 1983, Anal. Biochem. 132: 6-13) in order to prepare hybridization probes.

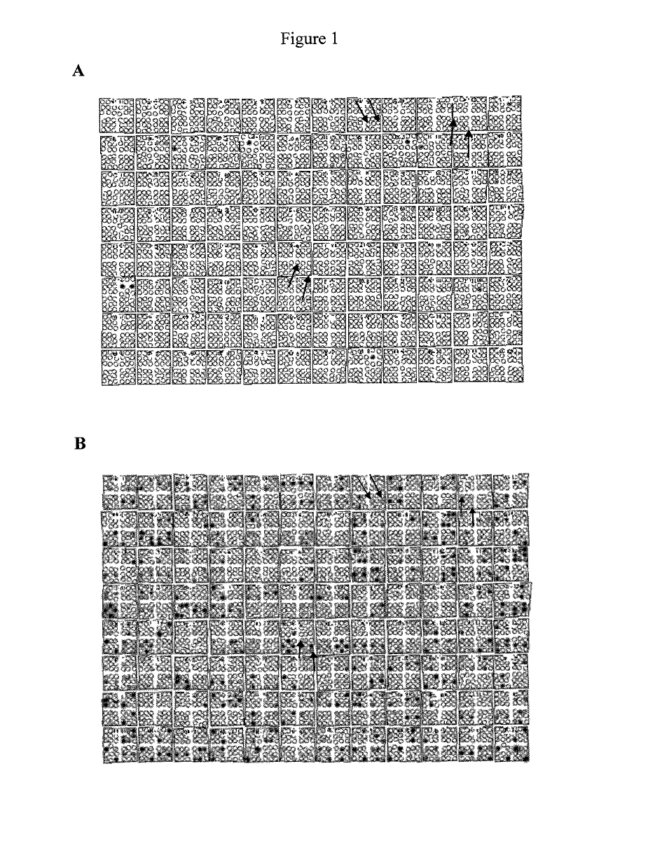

The membrane was preincubated in 25 ml of hybridization solution for 30 min at 65° C. (25 mM sodium phosphate, pH=7.5, 125 mM NaCl, 7% SDS). The hybridization probe was denatured at 100° C. for 10 min, then cooled on ice, about 100 CPM (“counts per minute”) per ml were added to the hybridization solution and the hybridization was carried out in a hybridization oven for 16 hours at 65° C. The membrane was then washed twice with the hybridization solution without probe at 65° C. for 10 min. The membrane was then washed at 65° C. a number of times for 10 min in each case in wash solution (2.5 mM sodium phosphate, pH =7.5, 12.5 mM NaCl, 0.7% SDS) until it was no longer possible to detect any activity in the solution poured off. The radioactive signals were analyzed using a phosphoimager (BioRad, Quantity One®) (FIG. 1) and subsequent analysis using an Array Vision 4.0 (Imaging Research Inc.). For this purpose, a mask was defined that was determined by the length and width of the area as well as the diameter of the spot positions. The autoradiography with an overlaid mask is indicated in FIG. 1. The standardization of the signal intensities was carried out by analyzing the positive control, that consisted of LPHR cDNA from yeast. This DNA was spotted on defined positions of the array and quantified by addition of a suitable probe to the hybridization probe of the wound specific or the skin specific cDNA pools. Those cDNAs were then selected which produced different standarized signal intensities with the various probes. This resulted at the positions of SW1368 on the membrane, in a significantly stronger signal intensity with the wound specific cDNA pool (FIG. 1B) in comparison with the skin specific cDNA pool (FIG. 1A). The sequencing of the clone (SEQ ID No. 15) and analysis of the sequence of 653 base pairs length showed, that it was a yet unknown GPCR.

### Example 2

### Verification of the Expression Pattern of SW1368 by means of , “Real-Time Quantitative RTPCR”

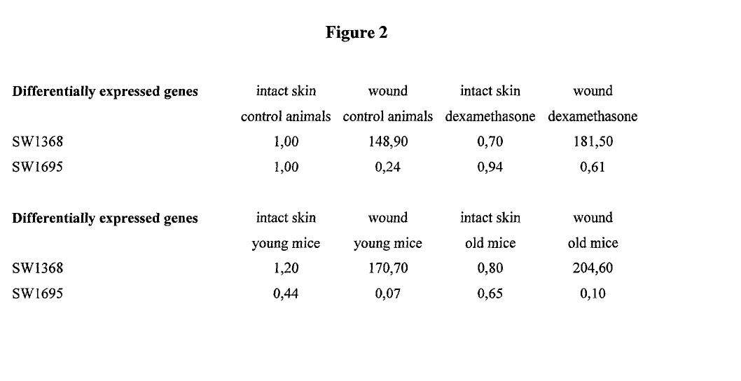

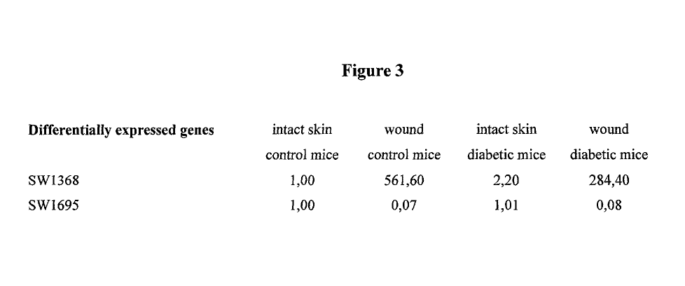

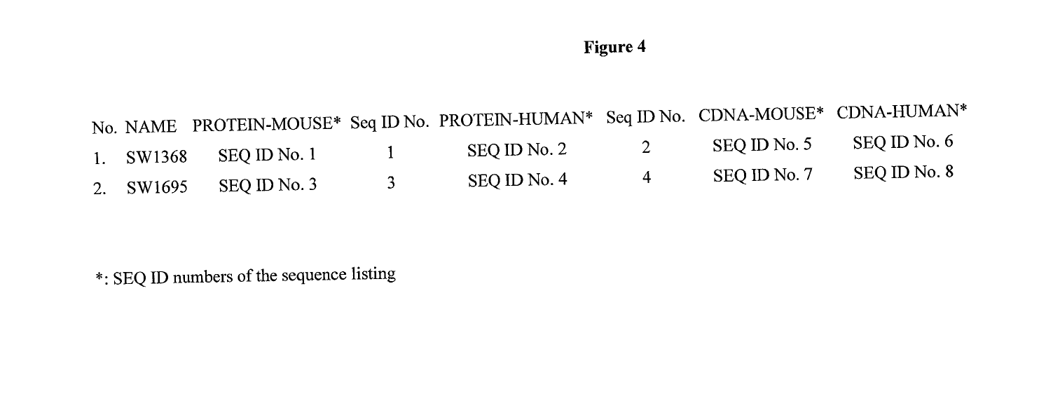

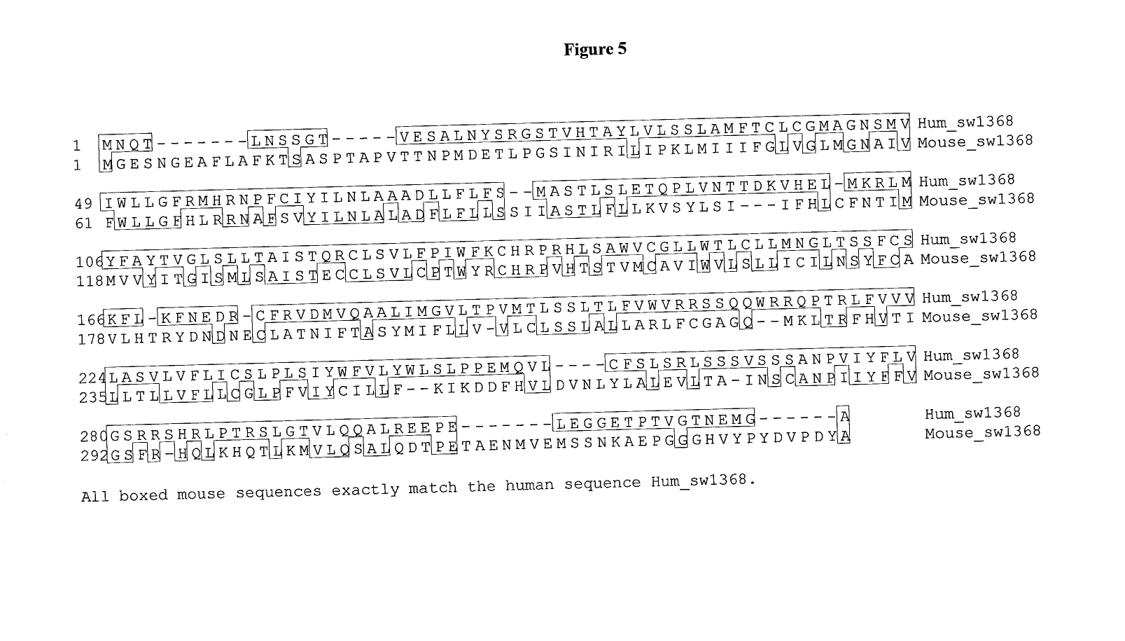

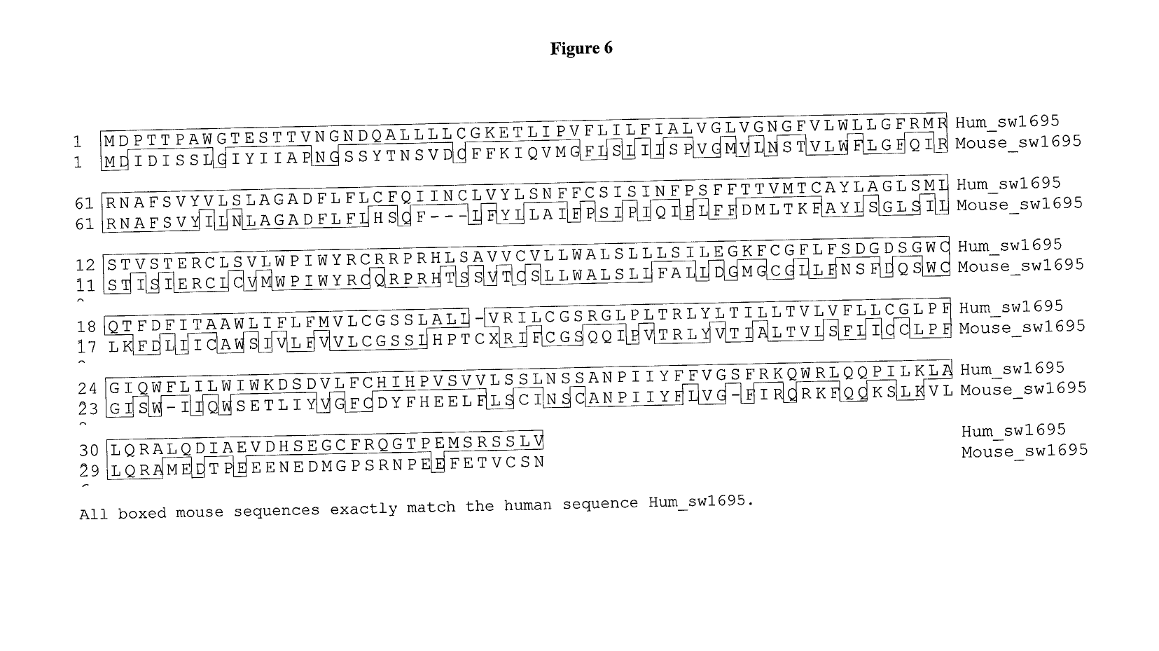

A verification of the differential expression of the nucleic acids described above as well as the investigation of further stages of wound healing was carried out by real-time RT-PCR in the ABI Prism 7700 sequence detection system (PE Applied Biosystems). The apparatus was equipped with the ABI Prism 7200/7700 SDS-Software Version 1.6.3 (1998). The detection of PCR products was carried out during the amplification of the cDNA with the aid of the stain SYBR Green 1, whose fluorescence is greatly increased by binding to double-stranded DNA (Karlsen et al. 1995, J. Virol. Methods. 55: 153-6; Wittwer et al., 1997, BioTechniques 22: 130-8, Morrison et al., 1998, BioTechniques 24: 954-62). The basis for the quantification is the PCR cycle (“threshold cycle”, CT value) which is reached when the fluorescence signal exceeds a defined threshold. The analysis is carried out by means of the Δ-CT method (User Bulletin #2, Relative Quantification of Gene Expression, PE Applied Biosystems, 1997). The abundances of the cDNAs were determined relative to an endogenous reference (GAPDH). The results are shown in FIGS. 2 and 3.

To obtain tissue from mice with poorly healing wounds, BALB/c mice were treated prior to wounding with dexamethasone (injection of 0.5 mg dexamethasone in isotonic salt solution per kg body weight twice a day for 5 days). To obtain wound tissue from mice with diabetes one-day-wounds of 10 weeks old C57BL/Ks-db/db/Ola mice were used. As control animals 10 weeks old C57BL/Ks were used in this case. Total RNA was obtained from skin and wound tissue as described above and 1 μg of total RNA was subjected to reverse transcription in a thermocycler (GeneAmp PCR system 9700, Perkin Elmer) using the TaqMan reverse transcription reagent kit (Perkin Elmer) according to the recomendations of the manufacturer (SYBR Green PCR and RT-PCR Reagents Protocol, PE Applied Biosystems, 1998). The primers for the amplification of the SW1368 cDNA SW1368-Primer 1: GAGGCATGTCAAATCAGTAAGCTG (SEQ ID No. 9), SW1368-Primer 2: GGTGGCTTTGGAGTGAGCAC (SEQ ID No.10) and the reference (GAPDH primer 1: ATCAACGGGAAGCCCATCA (SEQ ID No. 11), GAPDH primer 2: GACATACTCAGCACCGGCCT (SEQ ID No. 12)) were selected with the aid of the Primer Express software for Macintosh PC Version 1.0 (PE Applied Biosystems, P/N 402089, 1998) based on the CDNA sequence of SW1368 from mouse described above and the known sequence of GAPDH from mouse. For the PCR, the SYBR Green PCR Core Reagents Kit (4304886, PE Applied Biosystems) was used. The concentration of the primers in the PCR was initially optimized in the range from 50 nM to 600 nM and the specificity of the PCR was tested by analysis of the length of the amplified products in an agarose gel electrophoresis. The efficiency of the PCR system was then determined by means of a dilution series (User Bulletin #2, Relative Quantification of Gene Expression, PE Applied Biosystems, 1997). It became apparent that for both cDNAs the efficiency of the amplification was 100%, i.e. at each 1:2 dilution of the cDNA one more cycle was needed in order to exceed the fluorescence threshold value.

For the quantification, each batch of cDNA was amplified from 10 ng of reverse-transcribed total RNA in a total volume of 25 μl. The running conditions for the PCR corresponded to the details of the manufacturer (PE Applied Biosystems, SYBR Green® PCR and RT-PCR Reagents Protocol, 1998). The CT values were analyzed and therefrom the abundance of SW1368 mRNA relative to GAPDH was calculated. In the process both the increase of SW1368 in normally healing wounds in comparison with intact skin of control animals was confirmed as well as in wounds in comparison with skin of young and old mice (FIG. 2, compare FIG. 1). The analysis of further wound healing states showed, that expression SW1368 was slightly increased in poorly healing wounds of mice treated with dexamethasone in comparison with wounds of control animals. However, a 50% decreased amount of SW1368 could be shown in wounds of mice with diabetes in comparison to control mice (FIG. 3). Consequently, not only the wound specific regulation of SW1368 could be confirmed but additionally a role in disturbed wound healing could be shown (FIG. 3). In addition, it could be shown that SW1695 was specifically regulated in wounds (FIG. 2). In this case, a three fold higher amount of SW1695 was measured in poorly healing wounds of animals treated with dexamethasone than in wounds of control animals. This shows clearly that both SW1368 as well as SW1695 are regulated in different wound healing processes.

### Example 3

### Analysis of the Expression Pattern of SW1695 in Human Wounds

Skin biopsies (diameter: 4 mm) of untreated intact skin and of untreated one- and five-day wounds of healthy trial participants were removed by punching. These were examined by “real time quantitative RTPCR” as described in Example 2. The primers for the amplification of SW1695 cDNA (SW1695-Primer 1: TTCTTCTGCTTTGTGGCAAGG (SEQ ID No. 13), SW1695-Primer 2: GAAAAGGATCAGGAAGACCGG (SEQ ID No. 14) and the reference (hGAPDH-Primer 1: CATGGGTGTGAACCATGAGAAG (SEQ ID No. 16), hGAPDH-Primer 2: CTAAGCAGTTGGTGGTGCAGG (SEQ ID No. 17)) were selected based on the disclosed cDNA sequence of human SW1695 and based on the known sequence of human GAPDH. For the quantification cDNA derived from 10 ng reverse described total-RNA was amplified in a total volume of 25 μl for each sample. The PCR was carried out according to the specification of the manufacturer (PE applied biosystems, SYBR green PCR and RT-PCR reagents protocol, 1998). The CT values were analyzed and therefrom the abundance of SW1695 mRNA relative to GAPDH MRNA was calculated. A 60% decrease in SW1695 in one day wounds was measured in comparison with intact skin. This data is an accordance with the results obtained in the mouse model, where a comparable decrease in SW1695 was observed in normally healing wounds in comparison with intact skin of control animals (FIG. 2). In addition, a 70% decreased SW1695 expression could be shown in five day wounds in comparison with intact skin. This shows clearly that the expression of SW1695 is not only short-termed regulated but that an altered expression is essential for the healing process during a longer time period.

### Example 4

### Kinetics of Wound Healing of SW1368 Expression in Humans

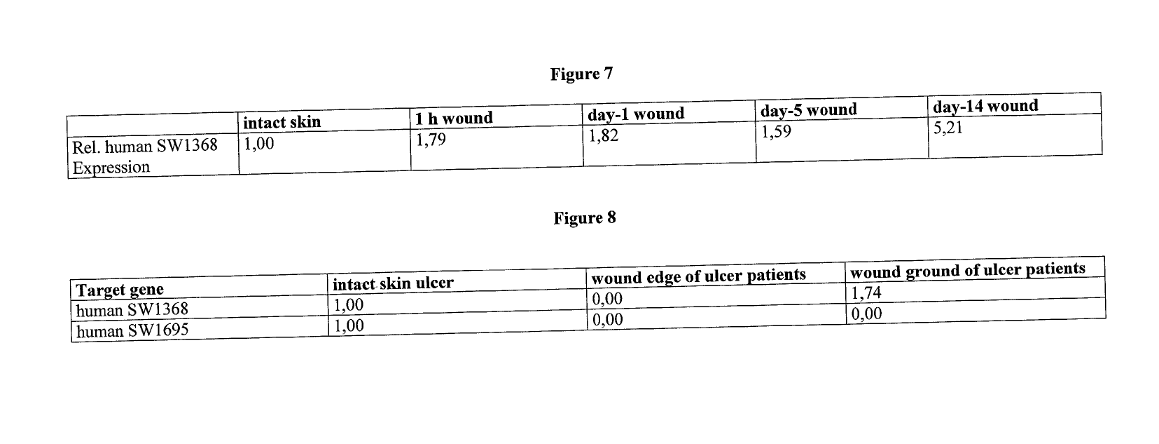

In order to obtain a better temporal resolution of the level of expression of GPCRs according to the invention during the wound healing process, 4 mm biopsies of intact skin of 6 patients were taken as described above, as well as biopsies at timepoint T=1 h, 1 d, 5 d, and 14 d. The biopsies of a given time point were pooled and the RNA was isolated. The RNA was isolated by homogenizing the biopsies in RNAclean buffer (AGS, Heidelberg), to which 1/100 part by volume of 2-mercaptoethanol had been added using a disperser. The RNA was then extracted by treating with phenol twice by means of acidic phenol saturated with water and extracted in the presence of 1-bromo-3-chloropropane. An isopropanol and an ethanol precipitation were then carried out and the RNA was washed with 75% ethanol. After this, a DNase I digestion of the RNA was carried out. For this, 20 μg of RNA (to 50 μl with DEPC-treated water) were incubated at 37° C. for 20 min with 5.7 μl of transcription buffer (Roche), 1 μl of RNase inhibitor (Roche; 40 U/μl) and 1 μl of DNase I (Roche; 10 U/μl). 1 μl of DNase I was then added again and the mixture was incubated at 37° C. for a further 20 min. The RNA was then treated with phenol, ethanol-precipitated and washed. All above mentioned steps were carried out using DEPC (diethyl pyrocarbonate)-treated solutions or liquids containing no reactive amino groups. cDNA was then prepared from the extracted RNA. This was carried out in the presence of 1×TaqMan RT buffer (Applied Biosystems), 5.5 mM MgCl₂ (Perkin Elmer), 500 μM each of dNTPs (Perkin Elmer), 2.5 μM of random hexamers (Perkin Elmer), 1.25 U/μl of MultiScribe Reverse Transcriptase (50 U/μl, Perkin Elmer), 0.4 U/μl RNase inhibitor (20 U/μl, Perkin Elmer), 20 μl of RNA (50 ng/μl) and DEPC-treated water (to 100 μl volume). After addition of the RNA and thorough mixing, the solution was divided in 2 0.2 ml wells (50 μl each) and the reverse transcription was carried out in a thermocycler (10 min at 25° C.; 30 min at 48° C. and 5 min at 95° C.). The cDNA was subsequently quantified by means of quantitative PCR using SYBR green PCR master mixes (Perkin Elmer), a triplicate determination (in each case with human SW1368 primers and cyclophilin primers) being carried out for each cDNA species to be determined. The stock solution for each triplet contained, in a total volume of 57 μl, 37.5 μl of 2×SYBR master mix, 0.75 μl of AmpErase UNG (1 U/μl) and 18.75 μl of DEPC-treated water. Per triplicate determination, 1.5 μl each of forward and reverse primer (hSW1368-Primer 1: GGAGTCAGCC CTAAACTATTCCAG (SEQ ID No. 20), hSW1368-Primer2: AGGTAGGCCG TGTGCACTGT (SEQ ID No. 21) were added to 57 μl of stock solution in a previously optimized concentration ratio. 60 μl each of the stock solution/primer mixture were mixed with 15 μl of cDNA solution (2 ng/μl) and subdivided into 3 reaction wells. Parallel to this, a stock solution with primers was prepared as a reference for the determination of cyclophilin (Cyclophilin-Primer 1:TCTTAACCAC CAGATCATTC CTTCT (SEQ ID No. 18) and Cyclo-philin-Primer 2: CCATAGTGCG AGCAAATGGG (SEQ ID No. 19)), mixed with a further 15 μl of the same cDNA solution and subdivided into 3 reaction wells. In addition, in order to set up a standard curve for the Cyclophilin-PCR, various cDNA solutions were prepared as a dilution series (4 ng/μl; 2 ng/μl; 1 ng/μl; 0.5 ng/μl and 0.25 ng/μl). 15 μl each of these cDNA solutions were mixed with 60 μl of stock solution/primer mixture for the determination of Cyclophilin and subdivided into 3 reaction wells. Likewise, a standard curve for the PCR of the human SW1368 gene to be investigated was set up in each case; the same dilutions which were also employed for the Cyclophilin standard curve were used here. The control used was a PCR batch without cDNA. 15 μl each of DEPC water were added to 60 μl in each case of stock solution/primer mixture of human SW1368 and Cyclophilin in each case, mixed and in each case subdivided into 3 reaction wells. The amplification of the batches was carried out in the GeneAmp 5700 (2 min at 50° C.; 10 min at 95° C., followed by 3 cycles of 15 sec at 96° C. and 2 min at 60° C.; then 37 cycles of 15 sec at 95° C. and 1 min at 60° C.). The analysis was carried out by the determination of the relative abundance of human SW1368 with respect to the Cyclophilin reference. For this, a standard curve was first set up by plotting the CT values of the dilution series against the logarithm of the amount of cDNA in the PCR batch (ng of transcribed RNA) and the slope(s) (s) of the straight lines was determined. The efficiency (E) of the PCR then results as follows: E=10^{-1/s}−1. The relative abundance (X) of the human SW1368 (Y) investigated in relation to Cyclophilin (cyc) is then: X=(1+_{Cyc})^{C}_{T}^{(CYc)}/(1+Ey)^{C}_{T}^{(y)}. The numerical values were then standardized by setting the amount of SW1368 cDNA from intact skin equal to 1. The results are compiled in FIG. 7. An up-regulation of the level of expression of SW1368 during wound healing observed in the mouse was confirmed in humans, the up-regulation of expression in humans being most prominent 14 days after wounding, significant but lower compared to the mouse. This demonstrates that the regulation of expression of GPCRs according to the invention is essential for wound healing.

### Example 5

### Dysregulated Expression of SW1368 and SW1695 in Human Ulcers

In order to show, that the genes SW1369 and SW1695 identified as relevant to wound healing play a role not only in normal wound healing but also in disorders of wound healing, biopsies of patients with chronic venous ulcers (ulcera venosum) were taken simultaneously from intact skin as well as from the wound ground as well as from the wound edge. and were analyzed for the level of expression of the target genes. For each group (intact skin, wound ground, wound edge) biopsies of 6 subjects were pooled. As described in Example 4 the RNA was isolated and cDNA was synthesized. The wound relevant cDNAs were quantified as described in Example 4, wherein the amount of Cyclophilin mRNA was used for the calculation of the relative amount of target gene cDNA. The results of the experiments are depicted in FIG. 8. For both genes a dysregulation of the level of expression in ulcers as compared to normal healing wound was observed (compare FIG. 7 and Example 3): in the case of SW1695 a significant but relatively small reduction of the level of expression by 60-70% was observed in day 1 and day 5 normal healing wounds relative to whereas SW1695 mRNA could not be detected in the wound ground or in the wound edge. This shows, that the dysregulation of SW1695 expression, especially the lack of mRNA can lead to severe wound healing disorders. In the case of SW1368 it could be shown that the mRNA is absent in the wound edge of venous ulcers, whereas in the case of normally healing wounds an upregulation of SW1368 expression was observed. In contrast to SW1695 a significant amount of SW1368 mRNA could be detected in the wound ground. The absence of both GPCRs according to the invention in the wound edge of ulcers, corresponding to the hyperproliferative epithelium of normally healing wounds shows that both genes participate in essential processes of wound healing, possibly during reepithelialization of proliferating keratinocytes of the hyperproliferative epithelium. This indicates, that the activity and/or expression of the GPCRs according to the invention has to be altered in pathological wound healing disorders, preferably activity and/or expression has to be increased. Preferably, the expression and/or activity has to be altered locally in the skin and/or wound.

### Example 6

### Differential Expression of Human SW1695 in Skin of Psoriasis Patients

It should now be verified with the aid of psoriasis patients that genes according to the invention play an important part not only in wound healing and wound-healing disorders but also in skin diseases. For this, 4 mm punch biopsies both of lesional and non-lesional skin were taken from psoriasis patients as described in Example 4. As a control, biopsies of intact skin were taken from healthy subjects. The isolation of the mRNA from the individual biopsies was carried out by embedding the biopsies in tissue freezing medium (Jung), the reduction of the biopsy into pieces using a microtome and the subsequent mRNA isolation by means of Dynabeads-Oligo dT (Dynal). The hackled biopsies were first suspended in lysis-buffer and then homogenized using the Polytron homogenizer. In order to fragment the genomic DNA, the solution is centrifuged through Qia-Shredder columns (Qiagen) and additionally sheared a number of times in a syringe with a needle. The Dynabeads were pretreated according to the instructions of the manufacturer and mixed with the lysis homogenate (250 μl of the stock suspension), incubated and washed (final volume 250 μl). The suspension was then divided into one portion each of 240 μl and of 10 μl (as a control). For the first strand synthesis, the following components were mixed: 20 μl of 10×TaqMan RT buffer, 44 μl of 25 mM MgCl₂, 40 μl of dNTP mix (2.5 mM/dNTP), 87 μl of DEPC-H₂O, 4 μl of RNase inhibitor (20 U/μl) and 5 μl of MultiScribe transcriptase (50 U/μl). 195 μl of the reaction mix were then added to the 240 μl batch and 20 μl to the control batch, mixed and incubated at 48° C. for 45 min. The Dynabeads were then pelleted in a magnetic particle collector and the supernatant was withdrawn. 20 μl of Tris-HCl buffer were then added and the suspension was incubated at 95° C. for 1 min. The Dynabeads were immediately pelleted in a magnetic particle collector and the mRNA in the supernatant was withdrawn. The cDNA/Dynabeads were then washed 3× with TE buffer. For the second strand synthesis, the cDNA/Dynabeads were washed 2× in 1× EcoPol buffer and a solution of the following components was added: 23 μl of 10× EcoPol buffer; 4.6 μl of dNTP mix (25 mM/dNTP); 11.5 μl of random hexamers; 118.7 μl of DEPC-H₂O. The suspension was mixed briefly with the aid of a vortexer and 9.2 μl of Klenow fragment (5 U/μl ) were then added. 200 μl of this solution were added to the batch, 20 μl to the control batch, and the suspensions were incubated at 37° C. for 1 h. The DNA was then melted at 94° C. for 1 min and the Dynabeads were pelleted in a magnetic particle collector. The supernatant was transferred to a new reaction vessel and the enzyme was inactivated at 75° C. for 10 min. The sense DNA strands contained in the supernatant were then employed for the TaqMan analysis.

The TaqMan analysis was carried out as described in Example 4, the amount of GAPDH (see Example 3) being used for the calculation of the relative abundance of the SW1695 mRNA in the individual biopsies. Since a far greater amount of total mRNA is isolatable from the skin biopsies of psoriasis patients, in particular from lesional skin, than from intact skin of healthy subjects, a standardization to identical amounts of mRNA is necessary, the amount of GAPDH mRNA being assumed as a housekeeping gene as a marker for the amount of total mRNA. A total of 2 biopsies of intact skin of healthy subjects were analyzed, and also 4 biopsies in each case of lesional and non-lesional skin from psoriasis patients. The abundances of SW1695 cDNA in the individual groups (intact skin, lesional skin, non-lesional skin) was then standardized to the total amount of the abundances of the cDNAs measured on a microtiter plate. Then, the average amounts of SW1695 mRNA was calculated per group (intact skin of healthy persons, non-lesional and lesional skin of psoriasis patients). Setting the amount of SW1695 mRNA to 1,00 in intact skin of healthy persons, the relative amount of SW1695 in unaffected, non-lesional skin of psoriasis is 3,80, whereas the amount in affected, lesional psoriatic skin is 1,11. This shows, that SW1695 can be used as a marker for psoriasis predisposition as unlesional skin displays an increased amount of SW1695 mRNA expression compared to skin of healthy persons. Also, the amount of SW1695 is decreased in lesional compared to non-lesional skin. Together, the data indicate, that dysregulation of genes or proteins according to the invention, especially of SW1695, can lead to skin disorders, in particular psoriasis. Hence, the expression and/or activity of SW1695 has perferentially to be altered for the prevention and/or treatment of skin disorders, in particular psoriasis. Preferentially, the expression and/or activity has to decreased for prevention of the development of lesions and has to be increased for the treatment of lesional skin.

Taken together this demonstrates that the differential expression of GPCRs according to the invention is not only essential for wound healing, but also that the dysregulation can lead to severe wound healing disorders and therefore the GPCRs according to the invention can be used for the diagnosis, prevention and/or treatment of diseases, for example of skin diseases, especially psoriasis and/or for diagnosis and/or treatment of diseases in wound healing and/or its pathological disorders.

It will be apparent to those skilled in the art that various modifications can be made to the compositions and processes of this invention. Thus, it is intended that the present invention cover such modifications and variations, provided they come within the scope of the appended claims and their equivalents.

Priority application DE 100381 11.1, filed Aug. 4, 2000 and priority application U.S. Ser. No. 60/229,501 filed Aug. 31, 2000. All publications cited herein are incorporated in their entireties by reference.

1. A G-Protein-coupled receptor-polypeptide according to SEQ ID No. 1 to SEQ ID No. 3.

2. A polypeptide according to claim 1, wherein the polypeptide is a fusion protein.

3. A nucleic acid encoding a polypeptide according to claim 1.

4. A nucleic acid according to claim 3, wherein the nucleic acid is contained in a knock-out gene construct, an expression construct, or in a vector selected from the group consisting of plasmids, shuttle vectors, phagemids, cosmids, expression vectors or vectors which are applicable in gene therapy.

5. A cell, containing a nucleic acid according to claim 3, or transformed with a vector, a knock-out gene construct or an expression construct according to claim 4.

6. A cell according to claim 5, wherein the cell is an heterologous or autologous cell, preferably a skin cell, a keratinocyte, a fibroblast or an endothelial cell.

7. A transgene embryonic non-human stem cell, wherein the stem cell contains a nucleic acid according to claim 3, preferably in the form of a vector, a knock-out gene construct or an expression construct containing the nucleic acid.

8. A transgenic non-human mammal, wherein its genome contains a nucleic acid according to claim 3, preferably in the form of a vector, a knock-out gene construct or an expression construct.

9. An antibody or an antibody fragment, wherein the antibody or antibody fragment is directed against a polypeptide according to claim 1 or against a nucleic acid according to claim 3.

10. A test for identification of pharmacologically active substances, wherein the test contains at least one polypeptide according to claim 1, and/or at least one nucleic acid encoding the polypeptide, and/or at least one antibody or an antibody fragment directed against the polypeptide, and/or at least one cell expressing the polypeptide or the nucleic acid encoding the polypeptide, and/or at least one transgenic non-human mammal containing the nucleic acid encoding the polypeptide, optionally combined or together with suitable additives or auxiliaries.

11. A test according to claim 10, wherein at least one polypeptide according to claim 1, and/or at least one nucleic acid encoding the polypeptide, and/or at least one antibody or an antibody fragment directed against the polypeptide, and/or at least one cell expressing the polypeptide or the nucleic acid encoding the polypeptide, is bound to a solid-phase.

12. An array fixated to a carrier material, wherein the array contains at least one polypeptide according to claim 1, and/or at least one nucleic acid encoding the polypeptide, and/or at least one antibody or an antibody fragment directed against the polypeptide, and/or at least one cell expressing the polypeptide or the nucleic acid encoding the polypeptide.

13. A diagnostic, wherein it contains at least one polypeptide according to claim 1, and/or at least one nucleic acid encoding the polypeptide, and/or at least one antibody or an antibody fragment directed against the polypeptide, and/or at least one cell expressing the polypeptide or the nucleic acid encoding the polypeptide, optionally combined or together with suitable additives or auxiliaries.

14. A diagnostic according to claim 13, wherein it contains a probe, preferentially a DNA probe.

15. A pharmaceutical, wherein it contains at least one polypeptide according to claim 1 and/or at least one nucleic acid encoding the polypeptide, and/or at least one antibody or an antibody fragment directed against the polypeptide, and/or at least one cell expressing the polypeptide or the nucleic acid encoding the polypeptide, optionally combined or together with suitable additives or auxiliaries.

16. A method of using at least one polypeptide according to claim 1, and/or at least one nucleic acid encoding the polypeptide, and/or at least one antibody or an antibody fragment directed against the polypeptide, and/or at least one cell expressing the polypeptide or the nucleic acid encoding the polypeptide, for production of a pharmaceutical preparation for diagnosis and/or prevention and/or treatment of skin disorders, and/or for treatment of wound healing and/or disorders of wound healing.

17. A method of producing a polypeptide, wherein a nucleic acid according to claim 1 is expressed in a suitable cell and, if appropriate, isolated.

18. A method of producing a transgene non-human mammal, wherein a transgenic embryonic non-human stem cell according to claim 7 is regenerated to a transgenic non-human mammal.

19. A method of producing an antibody or an antibody fragment, preferably a polyclonal or a monoclonal antibody or antibody fragment, wherein an antibody producing organism is immunized with a polypeptide according to claim 1 or with a nucleic acid encoding the polypeptide.

20. A method of producing a diagnostic or a pharmaceutical, wherein at least one polypeptide according to claim 1, and/or at least one nucleic acid encoding the polypeptide, and/or at least one antibody or an antibody fragment directed against the polypeptide, and/or at least one cell expressing the polypeptide or the nucleic acid encoding the polypeptide, is combined with suitable additives or auxiliaries.

21. A method of producing an array fixated to a carrier material, wherein at least one polypeptide according to claim 1, and/or at least one nucleic acid encoding the polypeptide, and/or at least one antibody or an antibody fragment directed against the polypeptide, and/or at least one cell expressing the polypeptide or the nucleic acid encoding the polypeptide, is fixated to said carrier material.

22. A method of using a test according to claim 10 for the identification of pharmacologically active substances in connection with diseases, preferably skin diseases, wound healing and/or disorders of wound healing.

23. A method of using an array according to claim 12 for analysis and/or diagnosis of diseases, preferably of skin diseases, wound healing and/or disorders of wound healing.

24. A method of using a G-protein coupled receptor-polypeptide according to the sequence of SEQ ID No. 4, and/or a nucleic acid encoding the polypeptide, and/or an antibody or antibody fragment directed against the polypeptide, and/or a cell containing the polypeptide or a nucleic acid encoding the polypeptide, and/or one fusion protein containing the polypeptide, for diagnosis, prevention and/or treatment of skin diseases, and/or for diagnosis and/or treatment in wound healing and/or disorders of wound healing.

25. A method of using a G-protein coupled receptor-polypeptide according to the sequence of SEQ ID No. 4, and/or a nucleic acid encoding the polypeptide, and/or an antibody or antibody fragment directed against the polypeptide, and/or a cell containing the polypeptide or a nucleic acid encoding the polypeptide, and/or a fusion protein containing the polypeptide, for identification of pharmacologically active substances in connection with skin diseases, wound healing and/or disorders of wound healing.

26. A method of using a G-protein coupled receptor-polypeptide according to the sequence of SEQ ID No. 4, and/or a nucleic acid encoding the polypeptide, and/or an antibody or antibody fragment directed against the polypeptide, and/or a cell containing the polypeptide or a nucleic acid encoding the polypeptide, and/or a fusion protein containing the polypeptide, for the production of a test for identification of pharmacologically active substances in connection with of skin diseases, wound healing and/or disorders of wound healing.

27. A method of using according to claim 26, wherein in order to produce the test, a polypeptide according to the sequence of SEQ ID No. 4, and/or a nucleic acid encoding the polypeptide, and/or an antibody or antibody fragment directed against the polypeptide, and/or a fusion protein containing the polypeptide, and/or a cell expressing the polypeptide or the nucleic acid encoding the polypeptide is bound to a solid phase.

28. A method of using a G-protein coupled receptor-polypeptide according to the sequence of SEQ ID No. 4, and/or a nucleic acid encoding the polypeptide, and/or an antibody or antibody fragment directed against the polypeptide, and/or a fusion protein containing the polypeptide, and/or a cell expressing the polypeptide or the nucleic acid encoding the polypeptide, for the production of an array fixated to a carrier material for the analysis of skin diseases, wound healing and/or disorders of wound healing.

29. A method of using according to one of claims 24 to 26 or 28, wherein the nucleic acid is contained in a knock-out gene construct, an expression construct, or in a vector selected from the group consisting of plasmids, shuttle vectors, phagemids, cosmids, expression vectors and vectors which is applicable in gene therapy.

30. A method of using according to one of claims 24 to 26 and 28, wherein the cell is an heterologous or autologous cell, preferably a skin cell, a keratinocyte, a fibroblast or an endothelial cell.

31. A method of using the pharmacologically active substances identified through a test according to claim 10 for manufacture of a pharmaceutical and/or diagnostic, wherein at least one pharmacologically active substances is combined with suitable auxiliaries and/or additives.

32. A method of using pharmacologically active substances identified through a test according to claims 10, for diagnosis, prevention and/or treatment of diseases, especially of diseases of the skin and for diagnosis and/or treatment of wound healing and/or disorders of wound healing.

33. A method of using according to at least one of claims 16, 22 to 28 and 31 to 32, wherein the disorder of wound healing is an ulcer of the skin, preferably a venous ulcer.

34. A method of using according to at least one of 16, 22 to 28 and 31 to 32, wherein the skin disease is psoriasis.
